(19) United States
(12) Patent Application Publication
(10) Pub. No.: US 2024/0166658 A1
(43) Pub. Date: May 23, 2024

(54) PORPHYRIN DERIVATIVES AND USES THEREOF

(71) Applicant: University of Tasmania, Sandy Bay (AU)

(72) Inventors: Nathan L. KILAH, Sandy Bay (AU); Chloe M. TAYLOR, Sandy Bay (AU); Michael C. BREADMORE, Sandy Bay (AU)

(21) Appl. No.: 18/278,694

(22) PCT Filed: Feb. 25, 2022
(86) PCT No.: PCT/AU2022/050155

§ 371 Date: Aug. 24, 2023

(30) Foreign Application Priority Data
Feb. 26, 2021 (AU) 2021900529

(51) Int. Cl. C07D 487/22; C09K 11/06; G01N 31/22; G01N 33/18; G01N 33/24; G01N 33/52

(52) U.S. Cl. CPC C07D 487/22; C09K 11/06; G01N 31/22; G01N 33/1826; G01N 33/24; G01N 33/52; C09K 2211/1074

(57) ABSTRACT

The present invention relates generally to porphyrin derivatives and their use in detecting poly- and perfluoroalkyl substances (PFAS). In particular, the present invention is directed to porphyrin derivatives having at least one receptor arm comprising an anion binding group substituted with a poly- or perfluorinated aliphatic group and their use as sensors for the detection of PFAS.

### RELATED APPLICATION

This application claims priority from Australian Provisional Patent Application No. 2021900529, the entire contents of which are incorporated herein by cross-reference.

### TECHNICAL FIELD

The present invention relates generally to porphyrin derivatives and their use in detecting poly- and perfluoroalkyl substances (PFAS). In particular, the present invention is directed to porphyrin derivatives having at least one receptor arm comprising an anion binding group substituted with a poly- or perfluorinated aliphatic group and their use as sensors for the detection of PFAS.

### BACKGROUND

Poly- and perfluoroalkyl substances (PFAS) are a group of potentially harmful molecules first created in the 1930s and include almost 5000 variations of synthetic fluoro-carbons. The strong fluorine-carbon bond (ca. 485 kJ mol⁻¹) gives PFAS many desirable physical properties for industrial and commercial production. In particular, PFAS were used extensively in the 1950s in large-scale manufacturing processes during the third industrial revolution due to their high degree of structural integrity. However, this structural integrity also leads to environmental longevity and a tendency of PFAS to bioaccumulate, giving rise to numerous environmental and health concerns.

The surfactant properties of PFAS meant they were used widely in firefighting foams, consumer goods like cookware, water resistant clothing and food packaging, among other everyday household items. In addition, industrial level usage has led to contamination of water and soil at numerous sites around the world. As such, most of the general public has been exposed to PFAS.

Of the almost 5000 different types of PFAS, there is a range of straight, branched and cyclic chain lengths having from 4-15 carbons. These molecules can breakdown in the environment to smaller PFAS, and a range of different size PFAS are typically present at any given contamination site. The physical characteristics of PFAS, such as solubility, hydrophobicity, and acidity, vary greatly with each additional carbon. The varying physical characteristics of PFAS means people can be exposed to PFAS through the respiratory, dermal, or digestive system. PFAS are not metabolised by the body, and the detrimental health impacts vary with chain length. Long chain perfluorinated carboxylic acids (PFCAs) are the most commonly observed in the environment (Land et al., 2018).

Perfluorooctanoic acid (PFOA) is a PFCA well-known from its extensive use in the manufacturing of products like Teflon®, Gore-Tex® and aqueous firefighting foams (AFFFs). PFOA is an eight-carbon chain perfluorinated carboxylic acid that is not produced in nature, yet is notably present in the blood serum of the majority of people living in industrialised countries (US median ca. 4 ng/mL; Gockener et al., 2020). PFOA has attracted significant attention by the media and regulatory bodies because of its relative detectability and high concentration being indicative of a broader range of PFAS. For example, the International Agency for Research on Cancer (IARC) classified PFOA as a class 2B substance in 2016 when exposure to PFOA was associated with cancers of the kidney, bladder, liver, pancreas, thyroid and prostate (Alexander and Olsen, 2007; Eriksen et al., 2009). Other studies have identified links between low birth weights and shown detrimental impacts on organ function when high levels of PFOA were measured in blood serum (Inoue et al, 2004; Jones et al., 2003; Emmett et al., 2006).

Since the regulation of PFOA, there has been an increasing occurrence of longer (C>9) PFCAs used in manufacturing, and consequently, an increased accumulation in the general population (Chambers et al., 2021). Multiple studies have suggested that long chain PFCAs are potentially more biologically harmful than the banned PFOA due to their increased bioaccumulation (Upham et al., 1998; Liao et al., 2009). Synthesis of other PFAS, known as GenX chemicals, also commenced in the late 1990s as an alternative to PFOA. However, such GenX chemicals—specifically the ammonium salt of hexafluoropropylene oxide dimer acid (HFPO-DA) fluoride (marketed as GenX™)—has since been shown to be toxic and carcinogenic.

Regulations to restrict the use of PFAS have been enforced by a number of countries to reduce any future contamination, but sites and sources of legacy PFAS are continuously being discovered. Current methods for PFAS detection require operator training and careful handling to avoid cross contamination or sorption of the analyte during the extraction processes. Such methods are costly, requiring sample extraction and preparation for testing using HPLC-MS, UPLC-MS, GC-MS, or LC/MS-MS, which can hinder remediation progress. For example, one of the more simple methods used for PFAS determination requires sonication of homogenised soil samples to be extracted using dispersive solid phase extraction and quantitation using isotope dilutions (Huset and Barry, 2018). This method is costly, labour intensive and only detects PFAS of 4-8 carbons long. The concurrent detection of PFCAs across a broad size range is analytically challenging, because often samples are of low concentrations in complex matrices. Further, a total oxidizable precursor assay (TOPA) cannot detect the entire size range of PFAS, as some will remain intact through the oxidation process. Many of the other techniques for PFAS detection developed still require pre-treatment or concentration of samples, a secondary analysis method for detection, are only semi qualitative or quantitative, are not selective or sensitive and/or cannot be useful for the limits of detection required for some regulations.

The number of potentially contaminated sites and water sources around the globe are driving the need for more rapid PFAS determination techniques. Accordingly, there is an ongoing need for improved or alternative methods for detecting the presence of PFAS, particularly for onsite detection of PFAS in biological and/or environmental samples, both pre- and post-remediation.

### SUMMARY

The present invention is predicated on the identification by the present inventors of porphyrin derivatives that are suitable for use as rapid, onsite photophysical sensors for the detection of a broad range of poly- and perfluoroalkyl substances (PFAS).

Accordingly, in a first aspect, the present invention provides a porphyrin derivative of Formula (I) or (II)

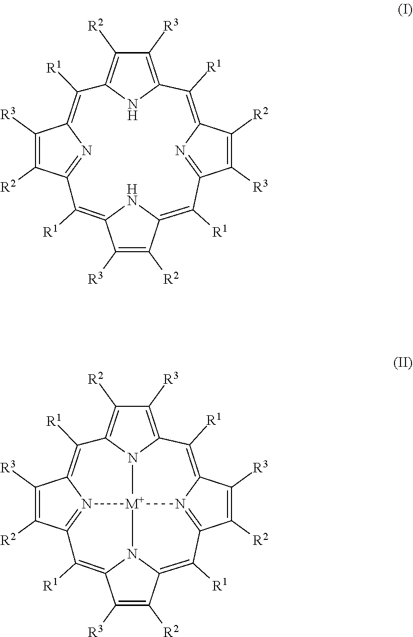

or a conformational isomer, stereoisomer and/or salt thereof, wherein
at least one R¹ is —A—R^{X}, C₆₋₁₀ aryl substituted with one or two —A—R^{X} groups or C₂₋₁₂ heteroaryl substituted with one or two —A—R^{X} groups, wherein
   each A is an anion binding group independently selected from the group consisting of a carbamate, toluene sulfonamide, amidourea, amide, NR′ (wherein R′ is selected from H and C₁₋₄ alkyl), ammonium, urea, thiourea, amido thiourea, guanidinium, squaramide and C₂₋₁₂ N-heteroaryl optionally substituted with one or more R^{Y} groups;
   each R^{X} is independently a straight chain, branched chain or cyclic C₁₋₂₀ poly- or perfluoroalkyl group, C₂₋₂₀ poly- or perfluoroalkenyl group, C₂₋₂₀ poly- or perfluoroalkynyl group or C₂₋₂₀ poly- or perfluoroalkylether group; and
   the remaining R¹ groups are independently selected from the group consisting of H, halo, CF₃, NH₂, NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₄ alkoxy, C(O)C₁₋₄ alkyl, C(O)OC₁₋₄ alkyl, C₆₋₁₀ aryl optionally substituted with one or more R^{Y} groups and C₂₋₁₂heteroaryl optionally substituted with one or more R^{Y} groups,
each R² and R³ is independently selected from the group consisting of H, halo, CF₃, NR″R″′ (wherein R″ and R′″ are independently selected from H and C₁₋₄ alkyl), NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₆ alkoxy, C(O)C₁₋₆ alkyl, C(O)OC₁₋₆ alkyl,
C₆₋₁₀ aryl optionally substituted with one or more R^{Y} groups, C₂₋₁₂ heteroaryl optionally substituted with one or more R^{Y} groups, and tri(C₆₋₁₀ aryl)borane optionally substituted with one or more R^{Y} groups or wherein R² and R³ together with the carbon atoms to which they are attached form a phenyl ring optionally substituted with one or more R^{Y} groups,
wherein each R^{Y} is independently selected from the group consisting of halo, CF₃, NR″R″′ (wherein R″ and R′″ are independently selected from H and C₁₋₄alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl; and
M⁺ is a metal ion.

In another aspect, the present invention provides a porphyrin derivative of Formula (I) or (II)

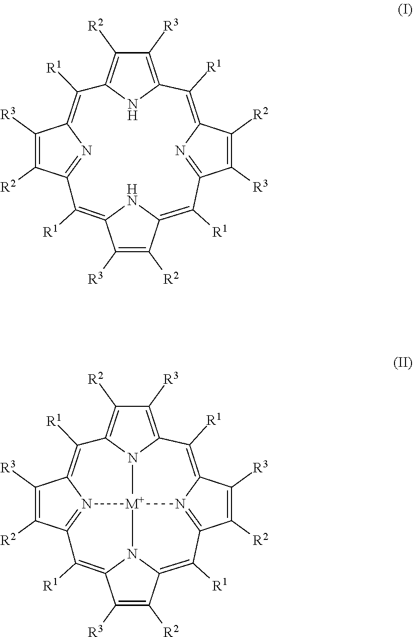

or a conformational isomer, stereoisomer and/or salt thereof, wherein
each R¹ is C₆₋₁₀ aryl substituted with one or two —A—R^{X} groups or C₂₋₁₂heteroaryl substituted with one or two —A—R^{X} groups, wherein
   A is an anion binding group selected from the group consisting of an amide, NR′ (wherein R′ is selected from H and C₁₋₄ alkyl), urea, thiourea and triazole;
   R^{X} is a straight chain, branched chain or cyclic C₃₋₁₅ poly- or perfluoroalkyl group or C₃₋₁₅ poly- or perfluoroalkylether group;
each R² and R³ is independently selected from the group consisting of H, halo, CF₃, NR″R″′ (wherein R″ and R′″ are independently selected from H and C₁₋₄ alkyl), NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₆ alkoxy, C(O)C₁₋₆ alkyl, C(O)OC₁₋₆ alkyl, C₆₋₁₀ aryl optionally substituted with one or more R^{Y} groups, C₂₋₁₂ heteroaryl optionally substituted with one or more R^{Y} groups and tri(C₆₋₁₀aryl)borane optionally substituted with one or more R^{Y} groups or wherein R² and R³ together with the carbon atoms to which they are attached form a phenyl ring optionally substituted with one or more R^{Y} groups,
wherein each R^{Y} is independently selected from the group consisting of halo, CF₃, NR″R″′ (wherein R″ and R″′ are independently selected from H and C₁₋₄ alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl; and
M⁺ is a metal ion.

The present invention also provides a porphyrin derivative of Formula (IA) or (IIA)

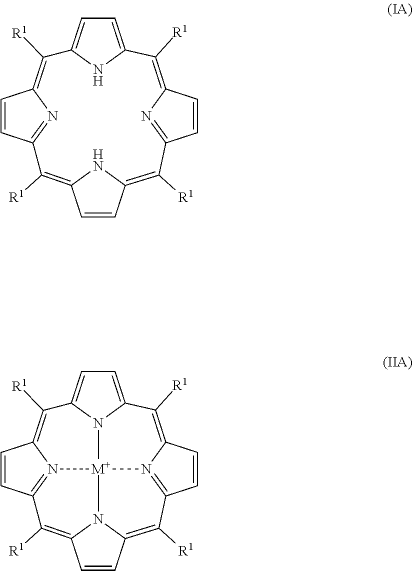

or a conformational isomer, stereoisomer and/or salt thereof, wherein
each R¹ is a phenyl group substituted with one —A—R^{X} group, wherein
   A is an anion binding group selected from the group consisting of an amide, NR′ (wherein R′ is selected from H and C₁₋₄alkyl), urea, thiourea and triazole;
   R^{X} is a straight chain, branched chain or cyclic C₃₋₁₅ poly- or perfluoroalkyl group; and
M⁺ is a metal ion.

In a preferred embodiment, the porphyrin derivative of Formula (I) or Formula (IA) is not the conformational isomer:

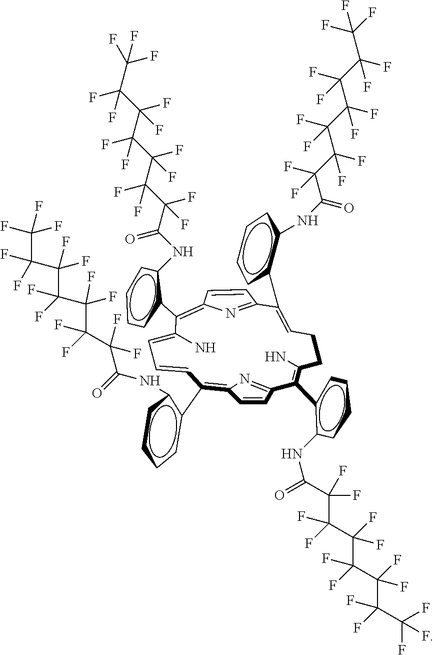

In another preferred embodiment, the porphyrin derivative of Formula (I) or (IA) is not:
α,α,α,α-5,10,15,20-tetrakis(heptafluorobutyramidophenyl)porphyrin;
α,α,α,α-5,10,15,20-tetrakis(2-(pentadecafluorooctanoylamino)phenyl)porphyrin;
α,α,α,β-5,10,15,20-tetrakis(2-(pentadecafluorooctanoylamino)phenyl)porphyrin;
α,α,β,β-5,10,15,20-tetrakis(2-(pentadecafluorooctanoylamino)phenyl)porphyrin;
6,20-Bis[4-(tridecafluoroheptanoylamino)phenyl]tetrabenzoporphyrin;
6,13,20,27-tetrakis[4-(tridecafluoroheptanoylamino)-phenyl]tetrabenzoporphyrin;
5,10,15,20-tetrakis(2-(2,3,3,3-tetrafluoro-2-(heptafluoropropoxy)propanoylamino)phenyl)porphyrin; or
α,α,α,α-5,10,15,20-tetrakis(heptafluorobutyramidophenyl)porphyrin.

In another preferred embodiment, the porphyrin derivative of Formula (II) or Formula (IIA) is not:
6,20-Bis[4-(tridecafluoroheptanoylamino)phenyl]tetrabenzoporphyrin platinum;
6,13,20,27-tetrakis[4-(tridecafluoroheptanoylamino)-phenyl]tetrabenzoporphyrin platinum;
5,10,15,20-tetrakis(2-(2,3,3,3-tetrafluoro-2-(heptafluoropropoxy)propanoylamino)phenyl)porphyrin cobalt; or
α,α,α,α-5,10,15,20-tetrakis(heptafluorobutyramidophenyl)porphyrin cobalt.

In another aspect, the present invention provides a host−guest complex comprising a porphyrin derivative of Formula (I) or Formula (II) as defined in the first aspect of the invention and a poly- or perfluoroalkyl substance (PFAS).

In another aspect, the present invention provides a method of detecting a poly- or perfluoroalkyl substance (PFAS) in a sample comprising the steps of:
(i) contacting a sample suspected of containing a PFAS with a porphyrin derivative of Formula (I) or Formula (II) as defined in the first aspect of the invention; and
(ii) detecting a photophysical change of the porphyrin derivative,
wherein a photophysical change of the porphyrin derivative indicates that the sample contains a PFAS.

In yet another aspect, the present invention provides use of a porphyrin derivative of Formula (I) or Formula (II) as defined in the first aspect of the invention as a PFAS sensor.

### BRIEF DESCRIPTION OF THE DRAWINGS

Embodiments of the invention will now be described with reference to the following Figures, which are intended to be exemplary only, and in which:

FIG. 1 shows the crystal structure of α,α,α,α-5,10,15,20-tetra-(2-amidophenyl-pentadecafluoro-octanoyl)porphyrin (9) in a dimeric lattice arrangement.

FIG. 2 is a UV-Vis spectrum of an anion screening of a host solution of an amide linked porphyrin derivative of the present invention (1×10⁻⁸ M) in dichloromethane combined with an aliquot containing ten molar equivalents of tetrabutylammonium (TBA) salts.

FIG. 3 is a UV-Vis spectrum of a titration of a host solution of an amide linked porphyrin derivative of the present invention in dichloromethane with tetrabutylammonium perfluorooctanoate (TBAPFO).

FIG. 4 shows the limits of detection of a host−guest complex of an amide linked porphyrin derivative of the present invention and perfluorooctanoic acid (PFOA) using fluorescence spectrophotometry; Excitation @ 449 nm, PMT @ 650V.

FIG. 5 is a UV-Vis spectrum of a titration of a host solution of a urea linked porphyrin derivative of the present invention in dichloromethane with a PFOA anion.

FIG. 6 is a UV-Vis spectrum of a titration of a host solution of a urea linked porphyrin derivative of the present invention in dichloromethane with perfluorooctanesulfonic acid (PFOS).

FIG. 7 shows the ¹⁹F NMR spectra of a urea linked porphyrin derivative of the present invention; (a) the urea linked porphyrin derivative (b) the urea linked porphyrin derivative bound to PFOS; and (c); the urea linked porphyrin derivative bound to PFOA.

FIG. 8 shows the averaged association constant (Kₐ) for the formation of a host−guest complex between an amide linked porphyrin derivative of the present invention and various PFAS of different carbon chain lengths.

FIG. 9 is a UV-Vis spectrum of a titration of a host solution of an amide linked short chain porphyrin derivative of the present invention in dichloromethane with PFOA.

FIG. 10 is a UV-Vis spectrum of a titration of a host solution of an amide linked long chain porphyrin derivative of the present invention in dichloromethane with PFOA.

FIG. 11 shows a comparison of shifts observed in the UV-Vis spectrum for amide linked short, medium and long chain porphyrin derivatives of the present invention with perfluorobutanoic acid.

FIG. 12 is a UV-Vis spectrum of an amide linked short chain porphyrin derivative of the present invention (2.01×10⁻⁶ M) combined with one equivalent of nine different perfluorocarboxylic acids (PFCAs) in dichloromethane.

FIG. 13 is a UV-Vis spectrum of an amide linked medium chain porphyrin derivative of the present invention (2.01×10⁻⁶ M) combined with one equivalent of nine different PFCA in dichloromethane.

FIG. 14 shows UV-Vis spectra for is a UV-Vis spectrum of an amide linked long chain porphyrin derivative of the present invention (2.01×10⁻⁶ M) combined with one equivalent of nine different PFCA in dichloromethane.

FIG. 15 shows a UV-Vis comparison of responses to perfluorododecanoic acid (PFDoDA) in the 650 nm region of amide linked short chain, medium and long chain porphyrin derivatives of the present invention.

FIG. 16 shows the red, green blue (RGB) response values for different host:guest ratios; inset shows that below a host:guest ratio of 1:1 there is significant difference between individual RGB response values.

FIG. 17 shows the RGB response values using a high concentration of guest.

FIG. 18 is a UV-Vis spectrum of the organic phase collected from “shake” vial extraction tests of a porphyrin derivative according to the present invention in dichloromethane combined with aqueous solutions of PFOA at 3 ppm.

FIG. 19 is a UV-Vis spectrum of an amide linked porphyrin derivative of the present invention before and after the addition of GenX™ (yellow) and PFOA (blue).

FIG. 20 shows ¹H NMR spectra showing shifts in an amide linked porphyrin derivative of the present invention in response to addition of GenX™.

### DEFINITIONS

Unless defined otherwise, all technical and scientific terms used herein have the same meanings as commonly understood by one of ordinary skill in the art to which this invention belongs.

Unless otherwise specified, the indefinite articles “a”, “an” and “the” as used herein, include plural aspects. Thus, for example, reference to “an agent” includes a single agent, as well as two or more agents; reference to “the composition” or “formulation” includes a single composition or formulation, as well as two or more compositions or formulations; and so forth.

As used herein, the term “about” means ±10% of the recited value.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps.

The term “consisting essentially of” means the inclusion of the stated integer or step or group of integers or steps, but other integer or step or group of integers or steps that do not materially alter or contribute to the working of the invention may also be included.

The terms “porphyrin derivative”, “porphyrin host”, “porphyrin sensor”, “PFAS sensor”, “host molecule”, “receptor” and the like are used interchangeably herein and are intended to have the same meaning.

As used herein, the term “receptor arm” or “arm” when used in relation to a porphyrin derivative refers to a moiety attached to the porphyrin base structure of the porphyrin derivative that is capable of binding a guest molecule via non-covalent interactions. The receptor arm(s) of the porphyrin derivatives disclosed herein comprise a poly- and perfluoroalkyl moiety. Preferably, a porphyrin derivative as disclosed herein comprises from 1 to 8 receptor arms, .e.g., 1, 2, 3, 4, 5, 6, 7 or 8 receptor arms. Where a porphyrin derivative has two or more receptor arms, each receptor arm may be the same moiety or they may be different moieties.

As used herein, the term “anion binding group” when used with reference to a host molecule (e.g., a porphyrin derivative) refers to a functional group or moiety that is capable of non-covalent binding to an anionic functional group or moiety on a guest molecule (e.g., a PFAS). Non-limiting examples of anion binding groups suitable for use in the present invention include a carbamate, toluene sulfonamide, amidourea, amide, NR′ (wherein R′ is selected from H and C₁₋₄alkyl), ammonium, urea, thiourea, amido thiourea, guanidinium, squaramide and C₂₋₁₂ N-heteroaryl or a salt thereof optionally substituted with one or more R^{Y} groups, wherein R^{Y} is selected from halo, CF₃, NR″R′″ (wherein R″ and R′″ are independently selected from H and C₁₋₄alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl. In some embodiments, the anion binding group is selected from benzimidazolium, halo imidazolium, halo triazole, halo triazolium, amidourea, triazole, triazolium, imidazole, imidazolium, amide, amine, ammonium, urea, thiourea, amido thiourea, pyrrole, pyridinium, pyridine, guanidinium, tetrazine, indole, carbazole, halo indole, halo carbazole or squaramide. Where a porphyrin derivative has two or more anion binding groups, each anion binding group may be the same or they may be different.

The general term “poly- and perfluoroalkyl substance(s)” (PFAS), as well as the names of specific PFAS, such as “perfluorooctanesulfonic acid” (PFOS), “perfluorooctanoic acid” (PFOA) and the ammonium salt of hexafluoropropylene oxide dimer acid (HFPO-DA) fluoride (GenX), are used throughout the specification to refer to the acid and conjugate base (anionic) form of the relevant PFAS depending on the context in which they appear. Acidic PFAS, such as PFOS and PFOA, may at least partially dissociate when dissolved in solution (e.g., in an aqueous and/or organic solvent). Thus, it will be apparent to those skilled in the art that the term PFAS encompasses the anionic form when present in solution (e.g., an aqueous solution, an organic solution or an aqueous/organic solution).

As used herein, the term “poly- or perfluorinated aliphatic group” refers to a straight, branched and cyclic aliphatic group or moiety. Preferably, the poly- or perfluorinated aliphatic group is an alkyl, alkenyl, alkynyl or alkylether group. The terms “polyfluorinated”, “polyfluoro” or the like when used in relation to an aliphatic group as disclosed herein refers to the carbon atoms of the aliphatic group being partially substituted with fluorine atoms, preferably two or more fluorine atoms. For example, a straight chain polyfluoroalkyl group will typically have at least one carbon atom substituted with two fluorine atoms (—CF₂—) or three fluorine atoms (—CF₃). The terms “perfluorinated”, “perfluoro” or the like when used in relation to, or as a prefix to, an aliphatic group as disclosed herein refers to the carbon atoms of the aliphatic group being completely substituted with fluorine atoms. For example, a straight chain perfluoroalkyl group as used herein will have the general formula —CₙF₂ₙ₊₁, where n is an integer from 1 to 20.

As used herein, the term “alkyl” refers to a monovalent (“alkyl”) and divalent (“alkylene”) straight chain or branched chain saturated aliphatic groups. The alkyl group may have from 1 to 20 carbon atoms, denoted C₁₋₂₀alkyl, or it may have from 3 to 15 carbon atoms, denoted C₃₋₁₅ alkyl, or it may have from 1 to 6 carbon atoms, denoted C₁₋₆ alkyl, or it may have from 1 to 4 carbon atoms, denoted C₁₋₄alkyl. Examples of suitable alkyl groups may include, but are not limited to, methyl, ethyl, 1-propyl, isopropyl, 1-butyl, 2-butyl, isobutyl, tert-butyl, amyl, 1.2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1.3-dimethylbutyl, 1,2,2-trimethylpropyl, 1,1,2-trimethylpropyl, heptyl, octyl, nonyl, decyl, undecyl, dodecyl, tridecyl, tetradecyl, pentadecyl, hexadecyl, heptadecyl, octadecyl, nonadecyl, icosyl and the like.

As used herein, the term “alkenyl” refers to a monovalent (“alkenyl”) and divalent (“alkenylene”) straight or branched chain unsaturated aliphatic hydrocarbon groups having at least one double bond anywhere in the chain. Unless indicated otherwise, the stereochemistry about each double bond may be independently cis or trans, or E or Z, as appropriate. The alkenyl group may have from 2 to 20 carbon atoms, denoted C₂₋₂₀ alkenyl, or it may have from 3 to 15 carbon atoms, denoted C₃₋₁₅ alkenyl, or it may have from 2 to 6 carbon atoms, denoted C₂₋₆ alkenyl, or it may have from 2 to 4 carbon atoms, denoted C₂₋₄ alkenyl. Examples of suitable alkenyl groups may include, but are not limited to, ethenyl, vinyl, allyl, 1-methylvinyl, 1-propenyl, 2-propenyl, 2-methyl- 1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, icosenyl and the like.

As used herein, the term “alkynyl” refers to monovalent (“alkynyl”) and divalent (“alkynylene”) straight or branched chain unsaturated aliphatic hydrocarbon groups having at least one triple bond. The alkynyl group may have from 2 to 20 carbon atoms, denoted C₂₋₂₀ alkynyl, or it may have from 3 to 15 carbon atoms, denoted C₃₋₁₅ alkynyl, or it may have from 2 to 6 carbon atoms, denoted C₂₋₆ alkynyl, or it may have from 2 to 4 carbon atoms, denoted C₂₋₄ alkynyl. Examples of suitable alkynyl groups may include, but are not limited to, ethynyl, 1-propynyl, 1-butynyl, 2-butynyl, 1-methyl-2-butynyl, 3-methyl-1-butynyl, 1-pentynyl, 1-hexynyl, methylpentynyl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl, icosynyl and the like.

As used herein, the term “alkylether” refers to monovalent and divalent straight or branched chain groups containing an oxygen or sulfur atom connected to two alkyl groups, wherein alkyl is as defined above and each alkyl group may be the same or different. The alkylether may have from 2 to 20 carbon atoms in total, denoted C₂₋₂₀ alkylether, or it may have from 3 to 15 carbon atoms in total, denoted C₃₋₁₅ alkylether, or it may have from 2 to 6 carbon atoms in total, denoted C₂₋₆ alkylether, or it may have from 2 to 4 carbon atoms in total, denoted C₂₋₄ alkyl.

As used herein, the term “alkoxy” refers to straight chain or branched alkoxy (O-alkyl) groups, wherein alkyl is as defined above. Examples of suitable alkoxyl groups may include, but are not limited to, methoxy, ethoxy, n-propoxy, isopropoxy, sec-butoxy, and tert-butoxy.

As used herein, the term “aryl” refers to an optionally substituted monocyclic, or fused polycyclic, aromatic carbocycle (i.e., a ring structure having ring atoms that are all carbon). The aryl group may have from 6-10 atoms per ring, denoted C₆₋₁₀ aryl. Examples of suitable aryl groups may include, but are not limited to, phenyl, naphthyl, phenanthryl. As used herein, the term “aryl” is also intended to encompass optionally substituted partially saturated bicyclic aromatic carbocyclic moiety in which a phenyl and a cycloalkyl or cycloalkenyl group are fused together to form a cyclic structure, such as tetrahydronaphthyl, indenyl or indanyl. The aryl group may be a terminal group or a bridging group.

As used herein, the term “cycloalkyl” refers to a saturated or partially saturated, monocyclic, fused or spiro polycyclic, carbocycle. The cycloalkyl group may have from 3 to 10 carbon atoms per ring, denoted C₃₋₁₀ cycloalkyl. Examples of suitable cycoalkyl groups may include, but are not limited to, cyclopropyl, cyclobutyl, cyclopentyl, cyclohexyl, spiro[3.3]heptanyl, decalin and adamantyl. The cycloalkyl group may be a terminal group or a bridging group.

As used herein, the term “cycloalkenyl” refers to a non-aromatic monocyclic or multicyclic ring system containing at least one carbon-carbon double bond. The cycloalkyl group may have from 5 to 10 carbon atoms per ring, denoted C₅₋₁₀ cycloalkenyl. Examples of suitable cycloalkenyl groups may include, but are not limited to, include cyclopentenyl, cyclohexenyl and cycloheptenyl. The cycloalkenyl group may be a terminal group or a bridging group.

As used herein, the terms “halogen” or “halo” are interchangeable and refer to fluorine, chlorine, bromine or iodine.

As used herein, the term “heterocycloalkyl” refers to a saturated or partially saturated, monocyclic, bicyclic, fused or spiro polycyclic carbocycles, wherein at least one (e.g., 1, 2, 3, 4 or 5) ring atom is a heteroatom independently selected from O, N, NH, or S. The heterocycloalkyl group may have from 2 to 6 carbon atoms per ring, denoted C₂₋₆ heterocycloalkyl. Examples of suitable heterocycloalkyl groups may include, but are not limited to, aziridinyl, azetidinyl, pyrrolidinyl, piperidinyl, piperazinyl, quinuclidinyl, morpholinyl, diazaspiro[3.3]heptane (e.g., 2,6-diazaspiro[3.3]heptane), tetrahydrothiophenyl, tetrahydrofuranyl and tetrahydropyranyl. The heterocycloalkyl group may be a terminal group or a bridging group and may be attached through a heteroatom or any carbon ring atom.

As used herein, the term “heteroaryl” refers to an optionally substituted monocyclic, or fused polycyclic, aromatic heterocycle, wherein at least one (e.g., 1, 2, 3, 4, 5, 6, 7 or 8) ring atom is a heteroatom independently selected from O, N, NH, or S. The heteroaryl group may have from 1-12 carbon atoms per ring, denoted C₁₋₁₂ heteroaryl. Examples of suitable heteroaryl groups include, but are not limited to, furyl, imidazolyl, isoxazolyl, isothiazolyl, oxadiazolyl, oxazolyl (e.g., 1,3-oxazolyl, 1,2-oxazolyl), pyridinyl (e.g., 2-, 3-, 4-pyridinyl), pyridazinyl, pyrimidinyl, pyrazinyl, pyrazolyl, pyrrolyl, tetrazolyl, thiadiazolyl, thiazolyl, thienyl, triazolyl (e.g., 1,2,3-triazolyl, 1,2,4-triazolyl), triazinyl, tetrazinyl and carbazolyl. Representative examples of bicyclic heteroaryl include, but are not limited to, benzimidazolyl, benzofuranyl, benzothienyl, benzoxadiazolyl (e.g., 2,1,3-benzoxadiazolyl), cinnolinyl, dihydroquinolinyl, dihydroisoquinolinyl, furopyridinyl, indazolyl, indolyl (e.g, 2- or 3-indolyl), isoquinolinyl (e.g., 1-, 3-, 4-, or 5-isoquinolinyl), naphthyridinyl (e.g., 1,5-naphthyridinyl, 1,7-naphthyridinyl, 1,8-naphthyridinyl, etc), pyrrolopyridinyl (e.g., pyrrolo[2,3-b]pyridinyl), quinolinyl (e.g., 2-, 3-, 4-, 5-, or 8-quinolinyl), quinoxalinyl, tetrahydroquinolinyl, and thienopyridinyl. In one or more embodiments the heteroaryl group is an N-heteroaryl group having one or more nitrogen heteroatoms, e.g., 1, 2, 3 or 4 nitrogen heteroatoms depending on the particular structure. N-heteroaryl groups may also have heteroatoms other than nitrogen, but N-heteroaryl groups are characterized by having at least one nitrogen heteroatom. Exemplary N-heteroaryl groups include imidazolyl, indolyl, (e.g., 2- or 3- indolyl), naphthyridinyl, pyrazinyl, pyridyl (e.g., 2-, 3- or 4-pyridyl), pyrrolyl, pyrimidinyl, quinolinyl (e.g., 2-, 3-, 4-, 5-, or 8-quinolinyl), isoquinolinyl, quinazolinyl, quinoxalinyl and triazinyl, benzimidazolyl, triazolyl, tetrazinyl and carbazolyl. As used herein, the term “heteroaryl” is also intended to encompass optionally substituted partially saturated bicyclic aromatic heterocyclic moiety in which a heterocycle and a cycloalkyl or cycloalkenyl group are fused together to form a cyclic structure. The heteroaryl group may be a terminal group or a bridging group and may be attached through a heteroatom or any carbon ring atom. The present invention is also intended to encompass salts of the N-heteroaryl groups disclosed herein. For example, the salt of an N-heteroaryl may be an acid addition salt, such as an HCl or HBr addition salt. Non-limiting examples of N-heteroaryl salts include benzimidazolium, imidazolium, triazolium and pyridinium salts.

As used herein, the term “optionally substituted” when used with reference to a particular group means that group may or may not be further substituted or fused (so as to form a polycyclic system), with one or more non-hydrogen substituent groups. Suitable optional substituents will be apparent to those skilled in the art. Exemplary optional substituents may include, but are not limited to, hydroxy, halo, nitro, azido, C₁₋₆ alkyl, C₂₋₆ alkenyl, C₂₋₆ alkynyl, C₁₋₆ alkoxy, C₃₋₁₀ cycloalkyl, C₅₋₁₀ cycloalkenyl, C₂₋₆ heterocycloalkyl, C₆₋₁₀ aryl, C₁₋₉ heteroaryl or CO(O)C₁₋₆ alkyl.

As used herein, the term “conformational isomer” or “conformer” refers to any two or more isomers that have the same molecular constitution that differ by rotation about one or more single bonds. Accordingly, the present invention encompasses porphyrin derivatives in substantially pure conformational form, as well as mixtures thereof. In some embodiments, the conformational isomer may be an “atropisomer” or mixture thereof. Atropisomers occur when steric or electronic hindrance to rotation about a single bond of a molecule causes a high enough barrier to interconversion of two conformers that individual conformers may be identified and/or isolated.

As used herein, the term “stereoisomer” refers to any two or more isomers that have the same molecular constitution and differ only in the three dimensional arrangement of their atomic groupings in space. Stereoisomers may be diastereoisomers, enantiomers or atropisomers. In some embodiments, the porphyrin derivatives described herein may contain asymmetric centres and are therefore capable of existing in more than one stereoisomeric form. Accordingly, the present invention encompasses porphyrin derivatives in substantially pure isomeric form at one or more asymmetric centres, e.g., greater than about 90% ee, such as about 95% or 97% ee or greater than 99% ee, as well as mixtures, including racemic mixtures, thereof.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### DETAILED DESCRIPTION

The present invention relates to porphyrin derivatives and their use in detecting perfluoroalkyl substances (PFAS). In particular, the present invention is directed to porphyrin derivatives having at least one receptor arm comprising an anion binding group substituted with a poly- or perfluorinated aliphatic group (e.g., a poly- or perfluorinated alkyl, alkenyl, alkynyl or alkylether group) and their use as PFAS sensors. The porphyrin derivatives disclosed herein may be particularly suitable for use as qualitative and/or qualitative sensors for the detection of PFAS, for example, in environmental or biological samples. In particular, the present inventors have found that the porphyrin derivatives of the present invention may provide a convenient onsite method for the rapid colorimetric detection of PFAS.

### Porphyrin Derivatives

Porphyrins (1) are a common structural motif in supramolecular chemistry for sensor molecules due to their strong absorbance and ability to accommodate modifications that enhance interactions with analytes, thereby enhancing selectivity and/or sensitivity. Porphyrins may be substituted at one or more meso- and β-positions on the ring to provide a porphyrin derivate. Further, porphyrin and their derivatives may exist in the free base form (1) or as a complex with various metal ions (2), for example, ions of alkali metals, alkaline earth metals, rare earth metals, actinides, transition metals, and post transition metals. Porphyrin derivatives according to the present invention encompass tetrabenzoporphyrin derivatives having base structure (1a) and metal complexes thereof.

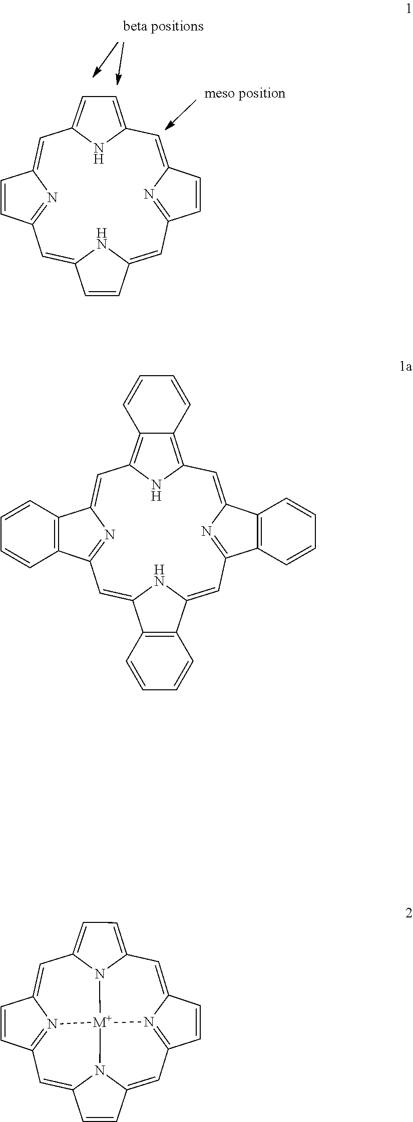

The present invention provides a porphyrin derivative comprising at least one (i.e., one, two, three or four) receptor arms. The receptor arms are formed by meso substitution(s) of the porphyrin base structure comprising an anion binding group further substituted with a poly- or perfluorinated aliphatic group. In some embodiments, the porphyrin derivative may have a picket fence conformation. As used herein, the term “picket fence” refers to a conformational arrangement in which the receptor arms or substituents at the meso positions on the porphyrin ring form a binding cavity for a guest molecule, such as a PFAS molecule, on one side of the ring. Thus, in preferred embodiments, the porphyrin derivatives of the present invention comprise at least one (i.e., one, two, three or four) receptor arms at the meso position comprising an anion binding group substituted with a poly- or perfluorinated aliphatic group on the same side of the porphyrin ring so as to provide a fluorine rich cavity for binding a PFAS guest molecule.

The receptor arms of the porphyrin derivatives of the present invention may comprise any suitable anion binding group known to those skilled in the art. For example, a variety of anion binding groups may be conveniently prepared from a porphyrin base structure substituted at one or more meso positions with a substituted C₆₋₁₀ aryl or substituted C₂₋₁₂ heteroaryl. Preferably, the porphyrin base structure substituted at one or more meso positions with a substituted phenyl group, e.g., a 2-subsituted phenyl. Advantageously, the use of a 2- (ortho-) substituted phenyl (e.g., a 2-aminophenyl) group at one or more meso position(s) of the porphyrin base structure provides a scaffold for forming a binding cavity suitable for PFAS in a 1:1 host−guest complex Similarly, the use of a 2,6-substituted phenyl group (or another appropriately di-substituted C₆₋₁₀ aryl or substituted C₂₋₁₂ heteroaryl) at one or more meso position(s) of the porphyrin base structure may provide access to a scaffold for forming two binding cavities (i.e., on both sides of the porphyrin ring) suitable for PFAS in a 1:2 host−guest complex. Further, the choice of ortho-substituent(s) can be tailored using techniques well known to those skilled in the art to provide access to a broad range of anion binding groups bearing a receptor arm. Exemplary anion binding groups that may be prepared from the meso substituted porphyrin base structure include, but are not limited to, a carbamate, toluene sulfonamide, amidourea, amide, NR′ (wherein R′ is selected from H and C₁₋₄ alkyl), ammonium, urea, thiourea, amido thiourea, guanidinium, squaramide, C₂₋₁₂ N-heteroaryl or a salt thereof optionally substituted with one or more R^{Y} groups, and C₁₋₂ alkylC₂₋₁₂ N-heteroaryl or a salt thereof optionally substituted with one or more R^{Y} groups, wherein R^{Y} is selected from halo, CF₃, NR″R′″ (wherein R″ and R′″ are independently selected from H and C₁₋₄ alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl. In some embodiments, the anion binding group is selected from an amine, amide, urea, thiourea and triazole. In a preferred embodiment, the anion binding group is an amide, which can be readily prepared, for example, from the 2-aminophenyl substituted porphyrin using standard techniques. Other suitable anion binding groups and processes for their preparation will be apparent to those skilled in the art.

In embodiments in which the porphyrin derivative of the present invention comprises one, two or three meso substitution(s) comprising an anion binding substituted with a poly-or perfluorinated aliphatic group (e.g., a poly- or perfluorinated alkyl, alkenyl, alkynyl or either group), the remainder of the meso positions may be unsubstituted or substituted with any suitable group known to those skilled in the art. Suitable substituent groups may include, but are not limited to halo, CF₃, NH₂, NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₄ alkoxy, C(O)C₁₋₄ alkyl, C(O)OC₁₋₄ alkyl, C₆₋₁₀ aryl optionally substituted with one or more R^{Y} groups and C₂₋₁₂heteroaryl optionally substituted with one or more R^{Y} groups. Suitable reactions for preparing meso-substituted porphyrin derivatives will be known to those skilled in the art and may include, for example, reactions disclosed in Figueira et al. Synthesis and anion binding properties of porphyrins and related compounds, Journal of Porphyrins and Phthalocyanines 2016, 20 (8), 950-965.

The anion binding group(s) of the porphyrin derivatives of the present invention are substituted with at least one poly- or perfluorinated aliphatic group (e.g., a poly- or perfluorinated alkyl, alkenyl, alkynyl or alkylether group). The poly- or perfluoroalkyl group(s) may be straight chain, branched chain or cyclic poly- or perfluorinated aliphatic group(s) being partially (poly-) or completely (per-) fluorinated. Preferably, the anion binding group is substituted with at least one perfluorinated aliphatic group. The perfluorinated aliphatic group may be C₁₋₂₀ straight chain, branched chain or cyclic poly- or perfluoroalkyl group or a C₂₋₂₀ straight chain, branched chain or cyclic perfluoroalkenyl, perfluoroalkynyl or perfluoroalkylether group(s). In some embodiments, the anion binding group is substituted with at least one C₃₋₁₅ straight chain, branched chain or cyclic perfluorinated aliphatic group, preferably a C₃₋₁₅ straight chain perfluorinated aliphatic group. In an embodiment, the anion binding group is further substituted with at least one C₇₋₈ straight chain perfluorinated aliphatic group, preferably a C₇₋₈ perfluorinated aliphatic group. In an embodiment, the anion binding group is further substituted with at least one C₉₋₁₂ straight chain perfluorinated aliphatic group, preferably a C₉₋₁₂ perfluorinated aliphatic group, more preferably a C₁₁₋₁₂ perfluorinated aliphatic group.

In some embodiments, the anion binding group(s) of the porphyrin derivatives disclosed herein are substituted with at least one alkylether group. Such alkylether groups may be incorporated into the porphyrin structure by nucleophilic substitution using a suitable poly- or per-fluoro alkylether carboxylic acid (via the corresponding acyl chloride) or poly-or per-fluoro epoxide (e.g., hexafluoropropylene oxide). Suitable reagents and reaction conditions will be apparent to those skilled in the art.

Suitable reagents and reaction conditions for incorporating the anion binding group(s) disclosed herein into the porphyrin structure will also be apparent to those skilled in the art. For example, carbamate groups may be incorporated into the porphyrin structure by the reaction of a suitable isocyanate reagent with an alcohol, conversion of a suitable hydroxamic acid, or the reaction of phosgene with suitable alcohols. Toluene sulfonamide groups may, for example, be incorporated into the porphyrin structure by nucleophilic substitution of a suitable sulfonyl chloride reagent, particularly poly or perfluorinated phenylsulfonyl chloride. Urea or thiourea groups may, for example, be incorporated into the porphyrin structure by the generation of a suitable isocyanate or isothiocyanate reagent and reaction with a suitable amine, conversion of a suitable hydroxamic acid, the addition of phosgene for an isocyanate, or the addition of carbon disulfide and lead nitrate, or carbon disulfide and N,N′-dicyclohexylcarbodiimide, to a poly or perfluoroalkylamine for isothiocyanates. Guanidinium groups may be incorporated into the porphyrin structure, for example, by reaction of an aminoporphyrin with a suitable carboxamidine reagent, such as N,N′-bis(tert-butoxycarbonyl)-1H-pyrazole-1-carboxamidine. Squaramide groups may be incorporated into the porphyrin structure, for example by nucleophilic substitution of a suitable mono poly- or perfluoroalkyl substituted squaramide reagent (e.g., 3-chloro-4-(perfluoroamino)-3-cyclobutene-1,2-dione) with an aminoporphyrin.

The β-positions of a porphyrin derivative of the present invention may be unsubstituted or one or more β-positions may be substituted with any suitable group known to those skilled in the art. Suitable substituent groups may include, but are not limited to, H, halo, CF₃, NR″R′″ (wherein R″ and R′″ are independently selected from H and C₁₋₄ alkyl), NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₆ alkoxy, C(O)C₁₋₆ alkyl, C(O)OC₁₋₆ alkyl, C₆₋₁₀ aryl optionally substituted with one or more R^{Y} groups, C₂₋₁₂ heteroaryl optionally substituted with one or more R^{Y} groups and tri(C₆₋₁₀ aryl)borane optionally substituted with one or more R^{Y} groups. Typically, β-substituents may be introduced via porphyrin condensation reactions using one or more functionalised pyrroles. These and other suitable methods for introducing β-substituents will be apparent to those skilled in the art.

In some embodiments, the pyrrole subunit of the porphyrin derivative may be fused to a phenyl group (i.e., to form an isoindole unit), so as to provide a tetrabenzoporphyrin base structure. The tetrabenzoporphyrin isoindole units may be further substituted, for example, via nucleophilic substitution with a variety of suitable nucleophiles. Suitable reagents and reaction conditions for generating a wide variety of substituted tetrabenzoporphyrins will be apparent to those skilled in the art.

The present invention also encompasses porphyrin derivatives as disclosed herein complexed to a metal ion. Suitable metals ions may include, but are not limited to, ions of alkali metals (e.g., lithium (Li), sodium (Na), potassium (K), rubidium (Rb), cesium (Cs) or francium (Fr)), alkaline earth metals (e.g., beryllium (Be), magnesium (Mg), calcium (Ca), strontium (Sr), barium (Ba) or radium (Ra)), rare earth metals (e.g., cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb) or yttrium (Y)), actinides (e.g., actinium (Ac), thorium (Th), protactinium (Pa), uranium (U), transition metals (e.g., scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), lanthanum (La), hafnium (Hf), tantalum (Ta), tungsten (W), rhenium (Re), osmium (Os), iridium (Ir), platinum (Pt), gold (Au), and mercury (Hg), and post transition metals (e.g., aluminium (Al), gallium (Ga), indium (In), tin (Sn), thallium (Tl), lead (Pb) or bismuth (Bi)). In an embodiment, the metal ion is selected from ions of Be, Mg, Ca, Sr, Ba, Ra, U, Sc, Ti, V, Cr, Mn, Fe, Co, Ni, Cu, Zn, Y, Zr, Nb, Mo, Ru, Rh, Pd, Ag, Cd, W, Re, Os, Ir, Pt, Au, Hg, Al, Ga, Sn, Tl, Pb or Bi. The identity and oxidation state of the metal may affect the photophysical properties of the porphyrin. As such, the choice of metal ion may, for example, effect any observable colour change or fluorescence response upon binding of the porphyrin derivative to PFAS.

Thus, in one aspect, the present invention provides a porphyrin derivative of Formula (I) or (II)

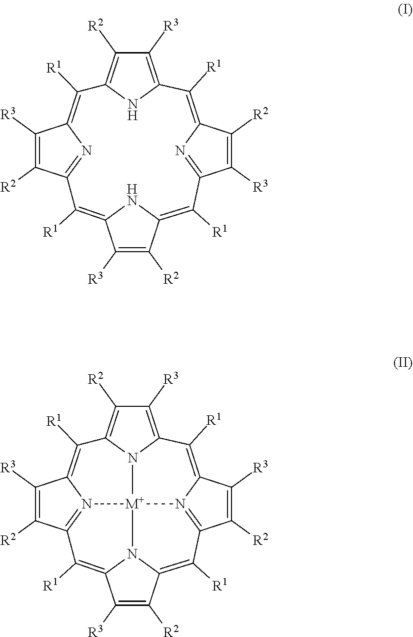

or a conformational isomer, stereoisomer and/or salt thereof, wherein
at least one R¹ is A—R^{X}, C₆₋₁₀ aryl substituted with one or two —A—R^{X} groups or C₂₋₁₂ heteroaryl substituted with one or two —A—R^{X} groups, wherein
   each A is an anion binding group independently selected from the group consisting of a carbamate, toluene sulfonamide, amidourea, amide, NR′ (wherein R′ is selected from H and C₁₋₄ alkyl), ammonium, urea, thiourea, amido thiourea, guanidinium, squaramide and C₂₋₁₂ N-heteroaryl or a salt thereof optionally substituted with one or more R^{Y} groups;
   R^{X} is a straight chain, branched chain or cyclic C₁₋₂₀ or poly- or perfluoroalkyl group, C₂₋₂₀ poly- or perfluoroalkylether group, C₂₋₂₀ poly- or perfluoroalkenyl group or C₂₋₂₀ poly- or perfluoroalkynyl group; and
   the remaining R¹ groups are independently selected from the group consisting of H, halo, CF₃, NH₂, NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₄ alkoxy, C(O)C₁₋₄ alkyl, C(O)OC₁₋₄ alkyl, C₆₋₁₀ aryl optionally substituted with one or more R^{Y} groups and C₂₋₁₂ heteroaryl optionally substituted with one or more R^{Y} groups,
each R² and R³ is independently selected from the group consisting of H, halo, CF₃, NR″R″′ (wherein R″ and R′″ are independently selected from H and C₁₋₄ alkyl), NO₂, CN, C₁₋₆ alkyl, C₁₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₆ alkoxy, C(O)C₁₋₆ alkyl, C(O)OC₁₋₆ alkyl, C₆₋₁₀ aryl optionally substituted with one or more R^{Y} groups, C₂₋₁₂ heteroaryl optionally substituted with one or more R^{Y} groups and tri(C₆₋₁₀aryl)borane optionally substituted with one or more R^{Y} groups or wherein R² and R³ together with the carbon atoms to which they are attached form a phenyl ring optionally substituted with one or more R^{Y} groups,
wherein R^{Y} is selected from halo, CF₃, NR″R″′ (wherein R″ and R′″ are independently selected from H and C₁₋₄ alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl; and
M⁺ is a metal ion.

In a preferred embodiment of Formula (I) or (II), or a conformational isomer, stereoisomer and/or salt thereof, each R¹ is C₆₋₁₀ aryl substituted with one or two —A—R^{X} groups or C₂₋₁₂ heteroaryl substituted with one or two —A—R^{X} groups, wherein
A is an anion binding group selected from the group consisting of an amide, NR′ (wherein R′ is selected from H and C₁₋₄ alkyl), urea, thiourea and triazole;
R^{X} is a straight chain, branched chain or cyclic C₃₋₁₅ poly- or perfluoroalkyl group or C₃₋₁₅ poly- or perfluoroalkylether group;
each R² and R³ is independently selected from the group consisting of H, halo, CF₃, NR″R″′ (wherein R″ and R′″ are independently selected from H and C₁₋₄ alkyl), NO₂, CN, C₁₋₆ alkyl, C₂₋₆ alkenyl, C₂₋₆ alkynyl, C₃₋₁₀ cycloalkyl, C₅₋₁₀ cycloalkenyl,
C₂₋₅ heterocycloalkyl, haloC₁₋₄ alkyl, C₁₋₆ alkoxy, C(O)C₁₋₆ alkyl, C(O)OC₁₋₆ alkyl, C₆₋₁₀ aryl optionally substituted with one or more R^{Y} groups, C₂₋₁₂ heteroaryl optionally substituted with one or more R^{Y} groups and tri(C₆₋₁₀ aryl)borane optionally substituted with one or more R^{Y} groups or wherein R² and R³ together with the carbon atoms to which they are attached form a phenyl ring optionally substituted with one or more R^{Y} groups,
wherein each R^{Y} is independently selected from the group consisting of halo, CF₃, NR″R″′ (wherein R″ and R″′ are independently selected from H and C₁₋₄ alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl; and
M⁺ is a metal ion.

In some embodiments of Formula (I) or (II), one, two, three or four R¹ groups are A—R^{X}, C₆₋₁₀ aryl substituted with one or two —A—R^{X} group or C₂₋₁₂ heteroaryl substituted with one or two —A—R^{X} group. In some embodiments, four R¹ groups are C₆₋₁₀ aryl substituted with one or two —A—R^{X} groups or C₂₋₁₂ heteroaryl substituted with one or two —A—R^{X} groups. In preferred embodiments, R¹ is C₆₋₁₀ aryl substituted with one —A—R^{X} group or C₂₋₁₂ heteroaryl substituted with one —A—R^{X} group. In an embodiment, R¹ is a phenyl group substituted with one —A—R^{X} group. Preferably, the phenyl group is substituted at the 2-position with one —A—R^{X} group. In another embodiment, the phenyl group is substituted at the 2- and 6-positions with two A—R^{X} groups.

Preferably, the porphyrin derivative of Formula (I) or Formula (II) has a picket fence conformation.

In some embodiments of Formula (I) or (II), A is selected from the group consisting of amino, urea and triazole.

In some embodiments of Formula (I) or (II), R^{X} is a straight chain C₃₋₁₅ poly- or perfluoroalkyl group, C₃₋₁₅ poly- or perfluoroalkenyl group, C₃₋₁₅ poly- or perfluoroalkynyl group or C₃₋₁₅ poly- or perfluoroalkylether. Preferably, R^{X} may be a straight chain C₇₋₈ poly- or perfluoroalkyl group, C₇₋₈ poly- or perfluoroalkenyl group or C₇₋₈ poly- or perfluoroalkynyl group. Preferably, R^{X} is a perfluoroalkyl group or perfluoroalkylether group.

In some embodiments of Formula (I) or (II), each R² and R³ is H.

In other embodiments of Formula (I) or (II), R² and R³ together with the carbon atoms to which they are attached form a phenyl ring (i.e., to provide a tetrabenzoporphyrin base structure).

In one embodiment, the present invention provides a porphyrin derivative of Formula (I) or Formula (II), wherein each R¹ is a phenyl group substituted with one A—R^{X} group, A is amino, urea or thiourea, R^{X} is a straight chain C₃₋₁₅ perfluoroalkyl group, each R² and R³ is H, and wherein the porphyrin derivative has a picket fence conformation.

The present invention also provides a porphyrin derivative of Formula (IA) or (IIA)

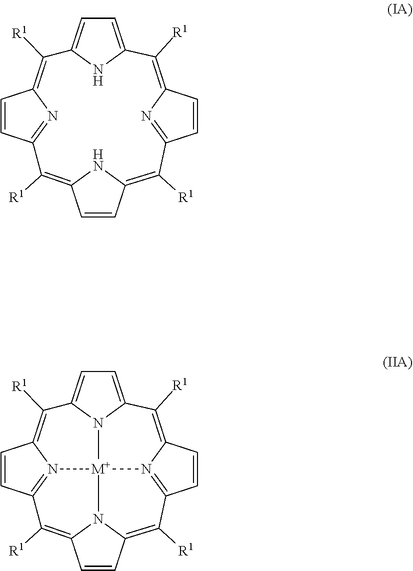

or a conformational isomer, stereoisomer and/or salt thereof, wherein
each R¹ is a phenyl group substituted with one —A—R^{X} group, wherein
   A is an anion binding group selected from the group consisting of an amide, NR′ (wherein R′ is selected from H and C₁₋₄alkyl), urea, thiourea and triazole;
   R^{X} is a straight chain, branched chain or cyclic C₃₋₁₅ poly- or perfluoroalkyl group or C₃₋₁₅ poly- or perfluoroalkylether group; and
M⁺ is a metal ion.

In preferred embodiments, the compounds of Formula (IA) and Formula (IIA) have a picket fence conformation.

In preferred embodiments of the compounds of Formula (IA) and Formula (IIA), R¹ as a phenyl group substituted at the 2-position with one —A—R^{X} group.

In preferred embodiments of the compounds of Formula (IA) and Formula (IIA), R^{X} is a straight chain C₃₋₁₅ poly- or perfluoroalkyl group or C₃₋₁₅ poly- or perfluoroalkylether group.

In some embodiments of the porphyrin derivative of Formula (II) or (IIA), R^{X} is a straight chain, branched chain or cyclic C₃₋₁₅ poly- or perfluoroalkyl group, preferably a straight chain C₃₋₁₅ poly- or perfluoroalkyl group.

In some embodiments, the porphyrin derivative of Formula (I) or (IA) is not:
α,α,α,α-5,10,15,20-tetrakis(heptafluorobutyramidophenyl)porphyrin;
α,α,α,α-5,10,15,20-Tetrakis(2-(pentadecafluorooctanoylamino)phenyl)porphyrin;
α,α,α,β-5,10,15,20-Tetrakis(2-(pentadecafluorooctanoylamino)phenyl)porphyrin;
α,α,β,β-5,10,15,20-Tetrakis(2-(pentadecafluorooctanoylamino)phenyl)porphyrin;
6,20-Bis[4-(tridecafluoroheptanoylamino)phenyl]tetrabenzoporphyrin;
6,13,20,27-tetrakis[4-(tridecafluoroheptanoylamino)-phenyl]tetrabenzoporphyrin;
5,10,15,20-tetrakis(2-(2,3,3,3-tetrafluoro-2-(heptafluoropropoxy)propanoylamino)phenyl)porphyrin; or
α,α,α,α-5,10,15,20-tetrakis(heptafluorobutyramidophenyl)porphyrin.

In some embodiments of the porphyrin derivative of Formula (II) or (IIA), M+ is a selected from the group consisting of an alkali metal, alkaline earth metal, rare earth metal, actinide, transition metal or post transition metal ion. Preferably, M is selected from the group consisting of zinc, iron, magnesium, manganese, nickel, titanium, chromium, cobalt or, copper ion.

In some embodiments, the porphyrin derivative of Formula (II) or (IIA) is not:
6,20-Bis[4-(tridecafluoroheptanoylamino)phenyl]tetrabenzoporphyrin platinum;
6,13,20,27-Tetrakis[4-(tridecafluoroheptanoylamino)-phenyl]tetrabenzoporphyrin platinum;
5,10,15,20-tetrakis(2-(2,3,3,3-tetrafluoro-2-(heptafluoropropoxy)propanoylamino)phenyl)porphyrin cobalt;
α,α,α,α-5,10,15,20-tetrakis(heptafluorobutyramidophenyl)porphyrin cobalt.

Porphyrin derivatives of Formula (I) or (II) may be prepared using any suitable methods known to those skilled in the art, the illustrative reaction schemes and general procedures disclosed herein, the specific methods described in the Examples, or by routine modifications thereof. The present invention also encompasses any one or more of the processes disclosed herein for preparing the porphyrin derivatives of Formula (I) or (II) and any novel intermediates used therein.

Suitable reagents and reaction conditions for performing the described reactions are known to the skilled person and are described in the literature and text books, including for example, Vogel, E. Novel porphyrinoid macrocycles and their metal complexes. J. Heterocyclic Chem., 1996, 33, 1461-1487, Vogel, E. The porphyrins from the ‘annulene chemist's’ perspective. Pure Appl. Chem., 1993, 65, 143-152; and Kadish, K., Smith, K. M. and Guilard, R. The Porphyrin Handbook, Volume 1, Elsevier Science: 2000.

The reaction schemes presented below are illustrative of general methods that may be employed to prepare the porphyrin derivatives of the invention. Alternative methods, including routine modifications of the methods disclosed herein, will be apparent to those skilled in the art.

A typical general synthesis for the preparation of porphyrin derivatives of Formula (I) is shown in Scheme 1, using α,α,α,α-5,10,15,20-tetra-(2-amidophenyl-pentadecafluoro-octanoyl)porphyrin as an exemplary porphyrin derivative of Formula (I). Suitable modifications to the reagents and reaction conditions to produce a broad range of porphyrin derivatives according to the present invention will be apparent to those skilled in the art.

The synthesis shown in Scheme 1 commences with the condensation of commercially available starting materials pyrrole (3) and 2-nitrobenzaldehyde (4) to form 5,10,15,20-tetrakis(2-nitrophenyl)porphyrin (5). This is followed by reduction of the nitro groups to provide 5,10,15,20-tetrakis(2-aminophenyl)porphyrin (6), which is converted to the α,α,α,α-5,10,15,20-tetrakis(2-aminophenyl)porphyrin (“picket fence”) isomer (7). A nucleophilic substitution reaction between the amine groups of isomer (7) and perfluorooctanoyl chloride (8) then forms α,α,α,α-5,10,15,20-tetrakis (2-(pentadecafluorooctanoylamino)phenyl)porphyrin (9).

Typically, in the first reaction depicted in Scheme 1,2-nitrobenzaldehyde is dissolved in glacial acetic acid and the solution heated to boiling with vigorous stirring. Pyrrole is then added dropwise, followed by refluxing for a suitable time (e.g., 30 min) to form 5,10,15,20-tetrakis(2-nitrophenyl)porphyrin (5). The precise period of time for any reaction disclosed herein may depend, for example, on the scale of the reaction and the particular reaction conditions, however those skilled in the art will be readily able to determine suitable time and temperature conditions, and will be able to monitor the progress of the reaction using standard techniques, such as Thin Layer Chromatography (TLC), ¹H NMR, etc to determine when the reaction is sufficiently or substantially complete. Once sufficiently complete, the reaction mixture is cooled to room temperature (e.g., below about 35° C.) with the dropwise addition of a suitable solvent (e.g., chloroform) and the solution allowed to stand for a suitable period of time for crystals to precipitate (e.g., about 6-18 h). The crystalline product may be isolated using standard techniques known to those skilled in the art, such as vacuum filtration and washing with a suitable solvent (e.g., chloroform), followed by drying of the crystals (e.g., at 100° C. for about 6-18 h).

It will be apparent to those skilled in the art that various modifications are possible to the first reaction depicted in Scheme 1 to provide access to a broad range of porphyrin derivatives of Formula (I). For example, use of a 3-substituted or 3,4-substituted pyrrole, or any combination of unsubstituted, 3-substituted and 3,4-substituted pyrroles would give access to a wide variety of β-substituted porphyrin derivatives of Formula (I). Where the pyrrole is a 3,4-subsituted pyrrole, each substituent may be the same or different. In some embodiments, the pyrrole may be fused to a phenyl ring (i.e., to provide an isoindole), which may be further substituted. Substituted pyrroles may be commercially available, or they may be synthesised using techniques known to those skilled in the art as discussed elsewhere herein. Additionally or alternatively, one, two or three arm porphyrin derivatives may be accessed by using a combination of 2-nitrobenzaldehyde (4) and other suitable aldehydes depending on the desired meso substituent (e.g., formaldehyde for H, acetaldehyde for methyl, and the like), as discussed elsewhere herein. Adjusting the reaction stoichiometry and conditions in order to achieve a one, two, three or four arm porphyrin derivative and/or the desired substitution pattern on the porphyrin ring is well within the purview of a person skilled in the art.

Typically, in the second reaction depicted in Scheme 1, porphyrin (5) is treated with reducing agent, such as tin(II) chloride hydrate in concentrated hydrochloric acid. The reaction mixture is stirred at room temperature about 90 min, following by heating (e.g., to about 65-70° C.) for a suitable time (e.g., about 25 min) to reduce the nitro groups to amines, providing 5,10,15,20-tetrakis(2-aminophenyl)porphyrin (6). Those skilled in the art know how to monitor the progress of a reaction using standard techniques, such as TLC, ¹H NMR, etc. The reaction mixture is then cooled and basified with a suitable base (e g , ammonium hydroxide to pH>10). The product may be isolated and purified using standard techniques known to those skilled in the art, such as solvent extraction, e.g., using an organic solvent such as chloroform, or the like, and washing with water and/or aqueous solution (e.g., ammonium hydroxide solution), as well as other well-known conventional techniques such as vacuum filtration. Porphyrin (6) may be purified, for example, using column chromatography using standard techniques. A suitable eluent for 5,10,15,20-tetrakis(2-aminophenyl)porphyrin (6) may be dichloromethane:methanol:triethylamine (10:0.5:0.001), however a skilled person will appreciate that the eluent may need to be adjusted depending on the nature of the porphyrin derivatives to be separated and will be able to adjust the eluent as a matter of routine.

In the third reaction depicted in Scheme 1, porphyrin (6) is optionally converted to the picket fence isomer by first heating a mixture of benzene and silica gel (e.g., to 80° C.) under nitrogen for a suitable amount of time to form a saturated atmosphere (e.g., two hours). Porphyrin (6) is then added and the mixture stirred while heating for an additional period of time sufficient to produce substantially the “picket-fence” (α,α,α,α-) isomer (e.g., about 20 hours) before cooling to room temperature. Extraction of the product may be achieved, for example, by pouring the reaction mixture onto a glass frit, washing with ether and benzene (1:1) to until a red colour change is visible in the filtrate, followed by eluting with acetone and ether (1:1) until the red colour is no longer visible in the filtrate, collecting the like fractions and removing the solvent. As porphyrin (7) tends to isomerise back to the statistical mixture (represented by structure (6)) while in contact with silica or in solution, any separation, isolation and/or purification steps are preferably performed in the absence of light (or under minimal light conditions).

In the final reaction depicted in Scheme 1, to synthesise the amide linked porphyrin derivative (9), picket fence porphyrin (7) (or optionally porphyrin (6)) is treated with a non-nucleophilic base (e.g., pyridine) in solvent (e.g., dichloromethane) for a suitable amount of time (e.g., one hour) prior to addition of pentadecafluorooctanoyl chloride (8), preferably dropwise in solution (e.g., dichloromethane). The reaction mixture is subsequently stirred at room temperature for a suitable amount of time until the reaction is sufficiently or substantially complete (e.g., 6-18 h). Those skilled in the art know how to monitor the progress of a reaction using standard techniques, such as TLC, ¹H NMR, etc. The product may be isolated and purified using standard techniques known to those skilled in the art, such as solvent extraction, e.g., using an organic solvent such as dichloromethane, or the like, and washing with water and/or aqueous solution (e.g., aqueous HCl, sodium hydrogen carbonate solution, brine), followed by column chromatography and/or recrystallisation.

It will be apparent to those skilled in the art that modifications to the final reaction in Scheme 1 may also provide access to a broad range of porphyrin derivatives of Formula (I). For example, the use of alternative straight chain, branched chain and cyclic poly- and perfluoroacyl chlorides provide access to the corresponding poly- and perfluoroalkyl substituents on the porphyrin ring. Such poly- and perfluoroacyl chlorides may be commercially available or may be synthesised using standard techniques known to those skilled in the art, such as by reacting the corresponding carboxylic acid with thionyl chloride (SOCl₂), phosphorous trichloride (PCl₃) or phosphorous pentachloride (PCl₅) under suitable conditions. Alternatively, different anion binding groups may be included at this stage. By way of non-limiting example, treatment of picket fence porphyrin (7) (or optionally porphyrin (6) as a mixture of atropisomers) with carbonyldiimidazole (CDI) in the presence of the desired poly- or perfluoralkyl hydroxamic acid provides access to the porphyrin derivative of Formula (I) or Formula (II) with a urea anion binding group (see, e.g., Usachova et al, 2010). Suitable methods for accessing other anion binding groups will be apparent to those skilled in the art.

A skilled person will appreciate that porphyrin derivatives of Formula (II) and (IIA) may be accessed via the same synthetic pathways as described above, with an additional step of incorporating a metal ion into the porphyrin ring. For example, a metal ion may be incorporated into the porphyrin ring by metallation. The type of metallation reaction may depend on the metal to be incorporated. For example, a typical zinc insertion reaction may involve stirring the relevant host molecule with an excess of zinc acetate in a solution of methanol and dichloromethane. Other suitable methods, which may depend on the specific metal ion to be incorporated, will be known to those skilled in the art. A skilled person will also appreciate that incorporation of a metal may occur at different stages in the synthetic pathway depending on the type of metal.

Porphyrin derivatives of the invention may be isolated or purified using standard techniques known to those skilled in the art. Such techniques include precipitation, crystallisation, recrystallization, column chromatography (including flash column chromatography), HPLC among others. Suitable solvents for use in these techniques will be known or can be readily ascertained by those skilled in the art using routine practices.

The above reactions are typically carried out in solution. Suitable solvent systems (including mixed solvent systems) are well known to those skilled in the art and those skilled in the art can readily select or determine a suitable solvent system using routine methods taking into consideration the nature of the porphyrin derivative of Formula (I) or Formula (II) and the amount of the porphyrin derivative of Formula (I) or Formula (II). Exemplary solvent systems include methanol, ethanol, water, acetone, tetrahydrofuran, dichloromethane, pentane, hexane, diethyl ether, ethyl acetate, and any mixture of two or more such solvents. Typically, porphyrin derivatives according to the present invention are crystalline. The crystalline products may precipitate out of solution and be collected by filtration or may be recovered by evaporation of the solvent, preferably in the absence of light and minimal heating.

### PFAS Sensors

The porphyrin derivatives according to the present invention may be suitable for use as qualitative, semi-quantitative and/or quantitative sensors for PFAS. In particular, the porphyrin derivatives of the present invention may produce a rapid colour change upon contact with PFAS, which may be discernible to the naked eye and/or by standard techniques known in the art, such as UV-Vis spectroscopy and fluorescence spectroscopy.

Environmental PFAS contamination sites such as water and soil can contain a wide variety of PFAS. All PFAS contain a chain of carbon atoms bonded to fluorine atoms. The carbon chain typically ranges from 4-15 carbons long and may be straight, branched or cyclic poly- or perfluoroalkyl chain. Some PFAS also have a functional group at the end of the carbon chain, such as carboxylic acid, sulfonic acid or sulfonamide Due to their stability, PFAS resist breakdown in the environment and bioaccumulate in living organisms. In particular, PFAS may bioaccumulate in humans due to exposure to and/or consumption of PFAS-containing materials (e.g., food and food packaging, commercial household products, fire-fighting foams and drinking water) and cause a variety of adverse health outcomes, including reproductive issues and low birth weight, various cancers (e.g., liver and kidney) and immunological disorders, among others. Perfluorocarboxylic acids (PFCAs), including PFOA, are a particularly prevalent subclass of PFAS. The chemical state of PFCAs is determined by temperature (MP/BP) and pKa and their distribution in the environment is influenced by their solubility, vapour pressure, and distribution coefficients.

The present inventors have found that porphyrin derivatives according to the present invention form a fluorine rich cavity that interacts with PFAS anions to produce a rapid colour change for a broad range of PFAS. Further, certain porphyrin derivatives disclosed herein may selectively bind PFAS in the presence of other anions. In particular, the porphyrin derivatives according to the present invention form host−guest complexes with PFAS, leading to an instantaneous photophysical change, preferably a colour change. The combination of non-covalent interactions between the receptor arm and the PFAS chain, and the anion binding group(s) with the PFAS anionic group, influence the conjugated electronic system of the porphyrin ring. These interactions result in a change in the photophysical properties of the porphyrin ring, which may be visualised as a colour change or detected using suitable detection methods (e.g., UV-Vis spectroscopy or fluorescence spectroscopy). The association constant and corresponding binding isotherm for various host−guest complexes prepared according to the present invention having a single binding cavity suggest the formation of a 1:1 host−guest complex. On this basis, host molecules having two binding cavities are expected form a 1:2 host−guest complex. Formation of the complex may occur under a variety of conditions, for example, in various organic solutions, aqueous extraction tests, dipstick tests and soil extraction tests, allowing for rapid onsite visual assessment of PFAS contamination. The porphyrin derivatives of the present invention may also be suitable for the detection of PFAS in biological samples, such as blood serum samples for rapid detection of PFAS bioaccumulation.

Thus, in one aspect, the present invention provides a method of detecting a poly- or perfluoroalkyl substance (PFAS) in a sample comprising the steps of: (i) contacting a sample suspected of containing a PFAS with a porphyrin derivative according to the present invention or a conformational isomer, stereoisomer thereof; and (ii) detecting a photophysical change of the porphyrin derivative, wherein a photophysical change of the porphyrin derivative indicates that the sample contains a PFAS.

In preferred embodiments, the photophysical change provides a visual colour change.

The porphyrin derivatives disclosed herein may be suitable for detection of PFAS in a sample from any site suspected of PFAS contamination. Typically, sites suspected of PFAS contamination include former industrial sites where PFAS and PFAS-containing products were manufactured, sites where firefighting foams that contained PFAS were used, and adjacent land, groundwater and surface water. Environmental samples that may be tested in accordance with the present invention include, for example, a fluid (e.g., surface water, groundwater, drinking water or waste water), soil, sludge, sediment, a solid surface (e.g., a fabric or food packaging), a biosolid, a plant, an animal (e.g., a marine animal in which PFAS is suspected of bioaccumulating) or any substance or material that is suspected of containing or coming into contact with PFAS.

In some embodiments, the methods according to the present invention may be used to determine whether a sample is free of PFAS. For example, the methods of the present invention may be used as a qualitative, semi-quantitative and/or quantitative method to determine whether remediation action has been effective. Thus, in some embodiments, testing may occur at existing remediation sites.

In an embodiment, the sample is an environmental sample, for example, a water sample or a soil sample.

In another embodiment, the sample may be a biological sample, for example, a blood serum sample.

In some embodiments, the porphyrin derivatives according to the present invention may be used to detect PFAS in a sample suspected of contamination without the need for any additional extraction or purification steps to isolate any PFAS, allowing for a range of versatile, inexpensive and rapid onsite visual field tests, which include, but are not limited to aqueous extraction tests, dipstick tests and soil extraction tests. Advantageously, the rapid detection of PFAS in a sample from an environmental site using the methods of the present invention may assist in the rapid remediation of environmental contamination sites. Further, the methods of the present invention may be used together with suitable techniques, such as UV-Vis spectroscopy or fluorescence spectroscopy, to provide a quantitative assessment of PFAS levels in a given sample.

The present inventors have also found that the presence of PFAS in a sample may be detected using colour space analysis, e.g., red, green, and blue (RBG) analysis, using an image obtained, for example, on a digital photography device, such as a smart phone, tablet or digital camera. By way of example, an image in the RGB colour space is composed of three data channels ranging from 0-255. A black object has an RGB value of (0,0,0), while a white object has an RGB value of (255,255,255). A digital photograph, for example, contains RGB information that can be extracted using commercially available software such as ImageJ or ColorX®, allowing an untrained observer to conduct rapid semi-quantitative chemical analysis for PFAS in a sample using porphyrin derivatives as disclosed herein and a simple digital imaging device, such as a smart phone. In some embodiments, total PFAS concentration is a sample may be estimated using a smart phone camera across a range of about 10 ppb (parts per billion) to about 16 ppm (parts per million), or above.

The methods according to the present invention comprise contacting a sample suspected of containing a PFAS (the PFAS sample) with a porphyrin derivative as disclosed herein. Preferably, the PFAS sample may be an aqueous solution or an aqueous extract, e.g., an aqueous soil extract, including an aqueous extract of a crude soil sample, or a solid material. The porphyrin derivative may be in any suitable form to enable contact with the PFAS sample. In some embodiments, the porphyrin derivative may be in an aqueous solution or water-miscible solution (e.g., water, DMSO, methanol, acetic acid, acetone, acetonitrile, ethanol), organic solution (e.g., dichloromethane, chloroform, toluene, diethyl ether, ethyl acetate, benzene, nitromethane), or a co-solvent mixture comprising any of the aforementioned solvents, among others. In other embodiments, the porphyrin derivative may be adsorbed onto a solid surface (e.g., a filter paper) or tethered to a solid surface using any suitable tether known in the art, and the solid surface carrying the porphyrin contacted with the PFAS sample, for example, by dipping the solid surface into the aqueous solution or dropping the PFAS sample onto the solid surface. Thus, contacting a PFAS sample with the porphyrin derivative may comprise combining the PFAS sample with the porphyrin derivative in a mono or biphasic solution, or contacting the PFAS sample with a solid surface comprising the porphyrin derivative. In preferred embodiments, the PFAS sample is an aqueous sample, while the porphyrin derivative may, for example, be in the form of an aqueous or organic solution or a solid sample. Accordingly, the methods of the present invention provide access to a versatile range of field tests, including but not limited to aqueous extraction tests (including biphasic “shake”), dipstick test and soil extraction tests, visual or colour space (e.g., RGB) analysis, allowing for rapid onsite assessment of PFAS contamination.

The present invention also provides use of a porphyrin derivative of Formula (I) or Formula (II) as a photophysical sensor for a PFAS. In preferred embodiments, the photophysical sensor is a colorimetric sensor.

The present inventors have found that the binding preference of the porphyrin derivatives of the present invention exhibited complementarity between PFAS length and the receptor arm length of the porphyrin derivative. For example, porphyrin derivatives with short poly- or perfluoroalkyl arm(s) tend to demonstrate stronger binding affinity (higher association constants, Kₐ) to short chain PFAS, with binding affinity tending to decrease as PFAS length increases. Similarly, porphyrin derivatives with a long poly- or perfluoroalkyl arm(s) tend to demonstrate stronger binding affinity (higher association constants, Kₐ) to long chain PFAS, with binding affinity decreasing as PFAS length decreases. Thus, it has been postulated that medium chain sensors (e.g., having a C₇₋₈ poly- or perfluoroalkyl chain) may be particularly suitable for detecting a broad size range of PFAS. It is expected that a similar effect would also be observed between PFAS and poly- or perfluoroalkenyl groups, poly- perfluoroalkynyl groups and poly- or perfluoroalkylether groups having a similar number of carbon atoms to the PFAS molecule.

Advantageously, the binding preference of porphyrin derivatives of the present invention having different receptor arm lengths may provide a method for selectively detecting PFAS of certain chain lengths in a sample, e.g., an environmental or biological sample. Different PFAS sizes are associated with different properties, toxicities and/or biological activities. For example, PFOA has been associated with a variety of cancers, low birth weights, detrimental impacts on organ function, decreased vaccine response, increased cholesterol, changes to liver enzymes, and preeclampsia. Thus, the porphyrin derivatives of the present invention may be suitable for use as targeted sensors for specific chain length PFAS to assist, for example, in remediation of environmental damage and/or diagnosis. Thus, in some embodiments, the receptor arm of the porphyrin derivative of the present invention (e.g., R^{X} in Formula (I) or (II)) is selected so as to be complementary to a PFAS suspected of being present in the sample.

As used herein the terms “complementary” or “complementarity” refers to the carbon chain length of the receptor arm of the porphyrin derivatives disclosed herein (e.g., R^{X} in Formula (I) or (II)) being similar to the carbon chain length of the PFAS suspected of being present in a sample. For example, the carbon chain length of the receptor arm may be within about ±3 carbons atoms of the carbon chain length of the PFAS, e.g., the carbon chain length of the receptor arm the PFAS may differ by about 3, preferably 2, more preferably 1, most preferably 0 carbon atoms. Preferably, the receptor arm is also similar in its structural arrangement to the PFAS suspected of being present in the sample. For example, a straight chain receptor arm may be preferably used to detect a straight chain PFAS. In particular, the presence of perfluorooctanesulfonic acid (PFOS) or perfluorooctanoic acid (PFOA) at environmental sites and in biological samples are indicative of PFAS contamination. Thus, in preferred embodiments, the receptor arm of the porphyrin derivative used in the methods of the present invention is a C₇₋₈ poly- or perfluorinated aliphatic group, more preferably a C₇₋₈ poly- or perfluoroalkyl or group or C₇₋₈ poly- or perfluoroalkylether or group, most preferably as straight chain C₇₋₈ poly- or perfluoroalkyl or group or C₇₋₈ poly- or perfluoroalkylether group.

As there are currently limited detection options available for short chain PFAS (i.e., having a chain length of less than 4 carbons), the use of complementary short arm porphyrin derivatives according to the present invention may be particularly useful for the detection of short chain PFAS contamination.

The porphyrin sensors disclosed herein may be suitable for the detection of PFCAs in unknown samples (i.e., in which the size of any PFAS present is unknown). In some embodiments, “long” chain sensors, e.g., having a C₉₋₁₅, preferably C₁₀₋₁₂, poly- or perfluoroalkyl chain, may be particularly suitable for the detection of PFAS in unknown samples because they can provide a more consistent response (both via UV-Vis absorption and colour space analysis) to a variety of PFAS chain lengths than the corresponding “short” (C₃₋₆) and “medium” chain (C₇₋₈) sensors. Thus, in one embodiment, the present invention provides a method for detecting PFAS in a sample comprising the steps of: (i) contacting a sample suspected of containing a PFAS with a porphyrin derivative according to the present invention or a conformational isomer, stereoisomer thereof, wherein R^{X} is a straight chain C₉₋₁₅ poly- or perfluoroalkyl group or C₉₋₁₅ poly- or perfluoroalkylether; and (ii) detecting a photophysical change of the porphyrin derivative, wherein a photophysical change of the porphyrin derivative indicates the presence of PFAS in the sample. Preferably, R^{X} is a straight chain C₁₀₋₁₂ poly- or perfluoroalkyl group or C₁₀₋₁₂ poly- or perfluoroalkylether.

In one aspect, the present invention also relates to a host−guest complex comprising a porphyrin derivative as disclosed herein and a PFAS. The PFAS may be any PFAS capable of forming a host−guest complex with a porphyrin derivation according to the present invention including, but not limited to carboxylic acid and sulfonic acid containing PFAS, and GenX. The host−guest complex may be a 1:1 complex or a 1:2 complex.

The present inventors have also found that certain porphyrin derivatives disclosed herein selectively bind PFAS in the presence of other anions including, but not limited to, Cl⁻, Br⁻, I⁻, HSO₄⁻, CH₃COO⁻, BF₄⁻. In particular, PFAS anion selectivity may improve with the number of “arms” on the porphyrin. Thus, in order to provide selective binding of PFAS in the presence of other ions, such as Cl⁻, Br⁻, I⁻, HSO₄⁻, CH₃COO⁻, BF₄⁻, the PFAS sensor is preferably a four arm porphyrin derivative, most preferably a four arm picket fence porphyrin derivative.

In some embodiments, the porphyrin derivatives according to the present invention produce a rapid colour change discernible to the naked eye upon contact with a PFAS in an aqueous solution at a concentration of less than about 50 ppm. For example, the limit of visual or digital RGB detection of a PFAS in aqueous solution using a porphyrin derivative according to the present invention may be less than about 50 ppm, or less than about 40 ppm, or less than about 30 ppm, or less than about 20 ppm, less than about 10 ppm, or less than about 5 ppm, e.g., about 5 ppm, 4 ppm, 3 ppm, 2 ppm, 1 ppm or less. In some embodiments, the limit of visual or colour space detection of a PFAS in aqueous solution using a porphyrin derivative according to the present invention may be less than 1 ppm, e.g., about 50 ppb, 40 ppb, 30 ppb, 20 ppb, or 10 ppb. The low limits of visual detection of PFAS make the porphyrin derivatives of the present invention particularly suitable as onsite PFAS sensors for environmental contamination (e.g., water and soil) sites. Lower limits of detection may also be achieved, for example, using fluorescence spectroscopy. For example, the limit of detection by fluorescence spectroscopy (e.g., 449 nm; PMT 650 V) of a PFAS in organic solution (e.g., dichloromethane) using a porphyrin derivatives according to the present invention may be less than about 500 ppb, or less than about 300 ppb, or less than about 100 ppb, or less than about 50 ppb, or less than about 10 ppb. Analytical techniques such as fluorescence spectroscopy and UV-Vis spectroscopy also provide a conventional method for the quantification of PFAS content in a given sample using standard calculations to determine the concentration of PFAS based on the fluorescence intensity or absorbance, respectively. Other suitable quantitative methods will be well known to those skilled in the art.

The present invention also encompasses kits comprising the porphyrin derivatives disclosed herein. The kits according to the present invention are preferably inexpensive, lightweight, readily transportable and/or easy to use, making them suitable for onsite use. The kits according to the present invention may comprise one or more components suitable for PFAS detection using an aqueous extraction test, biphasic extraction test, dipstick test, soil extraction test, or any other suitable test apparent to those skilled in the art. Thus, in an embodiment, the present invention provides a kit comprising a porphyrin derivative as disclosed herein and at least one solvent. The solvent may be an organic solvent (e.g., dichloromethane, chloroform), an aqueous solvent, or a combination thereof. The porphyrin derivative may be present in the kit in solid (e.g., crystalline) form, or pre-prepared in solution. For, example, the kit may comprise a vessel (e.g., a container, vial, tube or pouch) comprising the porphyrin derivative in solution, to which a sample suspected of contamination with a PFAS may be added directly. In another example, the kit may comprise a first vessel comprising the porphyrin derivative in solution and a second vessel to which the sample suspected of containing of containing a PFAS may be added, wherein the contents of the first and second vessel are subsequently combined, e.g., using a finger-actuated mechanism. In another embodiment, the present invention provides a kit comprising a porphyrin derivative as disclosed herein adsorbed onto a solid support (e.g., a dipstick) and a vessel for containing a sample suspected of contamination with a PFAS. In another embodiment, the present invention provides a kit comprising a porphyrin derivative bound to a solid particulate material (e.g., silica powder) in a vessel for passing through a sample suspected of contamination with a PFAS. In some embodiments, a kit according to the present invention may comprise two or more porphyrin derivatives having different size poly- or perfluorinated aliphatic group (e.g., a poly- or perfluorinated alkyl, alkenyl, alkynyl or alkylether group) receptor arms for the detection of different size PFAS. In other embodiments, a kit according to the present invention may comprise a porphyrin derivative comprising C₇₋₈ poly- or perfluorinated receptor arms, preferably C₇₋₈ poly- or perfluoroalkyl receptor arms. In other embodiments, a kit according to the present invention may comprise a porphyrin derivative comprising C₉₋₁₅ poly- or perfluorinated receptor arms, preferably C₉₋₁₂ poly- or perfluoroalkyl receptor arms.

In some embodiments, a kit according to the present invention may comprise a digital photography device connected to suitable software for RGB analysis, e.g., ImageJ or ColorX® (Benjamin Moore & Co.).

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples, which are intended for the purpose of illustration only and are not intended to limit the scope of the generality hereinbefore described.

### EXAMPLES

### Materials

Unless otherwise specified, proton (¹H), carbon (¹³C) and fluorine (¹⁹F) NMR spectra were recorded in (CD₃)₂SO, CDCl₃ or D₂O using a 600 MHz Bruker Avance 3 HD Narrow Bore Spectrometer (5 mm TCI tuneable probe) or a 400 MHz Bruker Avance 3 HD Wide Bore Spectrometer (5 mm BBFO probe) at room temperature (293 K) in CDCl₃ and C₆D₆. ¹H NMR spectra were obtained at 600.1 MHz and referenced to ¹H solvent resonance. ¹³C NMR spectra were obtained at 150.9 MHz and referenced to ¹³C solvent resonance. ¹⁹F NMR spectra were obtained at 376.0 MHz. NMR spectra were processed using Bruker Topspin 3.5 software. For ¹H NMR spectra, signals arising from the residual protio forms of the solvent were used as the internal standards. ¹H NMR spectroscopic data are recorded as follows: chemical shift (δ) [multiplicity, coupling constant(s) J (Hz), relative integral] where multiplicity is defined as: s=singlet; d=doublet; t=triplet; q=quartet; m=multiplet; br=broad or combinations of thereof. ¹H and ¹³C chemical shift values for spectra recorded in (CD₃)₂SO were referenced to residual dimethylsulfoxide (DMSO) appearing at δH=2.50 ppm and δC=39.52 ppm, respectively. ¹H and ¹³C chemical shift values for spectra recorded in D₂O were referenced to residual H₂O appearing at δH=4.79 ppm. ¹H and ¹³C chemical shift values for spectra recorded in CDCl₃ were referenced to residual CHCl₃ appearing at δH=7.26 ppm and the central resonance of the CDCl₃ “triplet” appearing at δC=77.16 ppm, respectively. ¹⁹F NMR chemical shift values for spectra recorded in CDCl₃ were referenced indirectly to DSS.

Fluorescence measurements were recorded using a Perkin Elmer LS-55 fluorescence spectrophotometer. Data for single crystal structures was obtained by X-ray diffraction collected using macromolecular crystallography MX1 and MX2 beamlines at the Australian Synchrotron and Bruker D8 Quest at the University of Tasmania. Structures were solved and refined using Olex² Crystallographic software. ESI−MS was performed using a Varian 1200 triple quadrupole mass spectrometer and a Thermo Scientific LTQ Orbitrap high resolution tandem mass spectrometer. IR analyses was performed using a Shimadzu FTIR-8400s Fourier Transform IR spectrometer and processed using Shimadzu IR Solution 1.60. UV-Vis analyses were performed using an LLG-UniSpec 2 Spectrophotometer and processed using MetaSpec Pro.

All reagents were purchased from Sigma-Aldrich or Combi-Blocks and were used as received. Deuterated solvents were purchased from Novachem and used as received. Analytical thin layer chromatography (TLC) was performed on aluminium-backed 0.2 mm thick silica gel 60 F254 plates. Eluted plates were visualized with a 254 nm UV lamp and/or by treatment with a suitable dip followed by heating. These dips included potassium permanganate/potassium carbonate/5% sodium hydroxide aqueous solution/water (3 g:20 g:5 mL:300 mL). Chromatographic separations were carried out according to protocols defined by Still et al. J. Org. Chem. 1978, 43, 2923 with silica gel 60 (40-63 μm) as the stationary phase and with the AR- or HPLC-grade solvents indicated.

Ideal materials to be used when preparing PFAS samples include polypropylene, high density polyethylene, PVC, stainless steel, and silicone. Some analysis methods require the use of materials that may adsorb PFAS (primarily glass). Glassware use was limited when possible, and it was acknowledged that it could have a minor impact on the effective PFAS concentration during analysis. Glassware that once contained PFAS material was not reused throughout experiments. Materials that must be avoided to limit PFAS contributions to analysis include low density polyethylene and polytetrafluoroethylene (Teflon®).

### General Procedures

Unless otherwise, specified, the following general procedures were employed in the following Examples.

### General Method of UV-Vis Titration

Path length of the quartz cell was 1 cm. For UV-vis titrations, a stock solution of host was prepared (2.2×10⁻³ M) in dichloromethane (DCM) and serially diluted to the required concentrations. Working solutions of tetrabutylammonium (TBA) anion salts or guest molecules were prepared in the same manner Host−guest titrations used solutions of guest prepared in the working concentration of host solution, according to the procedure of Thordarson, 2011.

### General Method for Determination of Association Constants (K)

UV-vis spectroscopic host−guest titrations were performed in accordance with the methodologies of Thordarson, 2011. Solutions of host in dichloromethane were prepared. Solutions of guest were then prepared in the host solution. Aliquots of guest in host solution were added to host solution and sequentially analysed until a known molar equivalent of guest was added. The amount of guest added per aliquot was ascertained by a preliminary screening experiment; strong association constants required smaller molar equivalents so more information could be collected. By way of example, host (9) responded to 0.1 molar equivalent of PFBA strongly, so an addition of 1 molar equivalent of PFBA would result in a loss of information. The inverse is true for weaker binding interactions. Molar equivalents between 0.1-5 typically provided adequate information for modelling. The data was then used to simulate binding isotherms using www.supramolecular.org (Hibbert and Thordarson, 2016). The data was fitted to 1:1, 1:2 and 2:1 equilibria making no assumptions about the cooperativity of the binding interactions and modelled using different algorithms. These experiments mainly used the Nelder-Mead (Simplex) method. The L-BFGS-B (quasi-Newtonian) method, which has higher importance/constraints on K value estimates, was also tested, and provided similar results unless stated otherwise. A model was excluded if it could not be successfully fit, or there was a significantly large error associated with the output.

### General Method for RGB Colour Analysis

A Puluz® 20 cm portable light tent with moderate and dispersed white LED lighting was used to photograph samples using an iPhone 6S plus camera on a fixed tripod. The automatic flash settings were disabled so there was no reflective interference on the sample vials. Samples that were being directly compared were photographed together to ensure lighting conditions and settings were consistent. The photographs were analysed using ImageJ software according to the procedure of Gallagher, 2014b. Multiple RGB values were chosen from areas of each sample at random to provide an “average” RGB value (Menesatti et al., 2012; Gallagher, 2014a; Phuangsaijai et al., 2021). Those RGB values were used to produce an artificial colour tile for visual comparison, or parameterized for modelling.

To quantify a colour difference, RGB values were transformed within the CIELab colour space. The difference, expressed as ΔE, was determined by measuring the relative distance between two colours. The numeric ΔE value can be used to predict how the two colours are perceived by a standard observer; a ΔE>2 is considered the minimum value to achieve a “just noticeable difference” (JND) (Table 1), which is the smallest difference required for an untrained observer to be able to determine two colours as being different (Castillo et al., 2021).

TABLE 1
ΔE values and corresponding perceived colour
differences as interpreted by an untrained observer

| ΔE | Observation |
|---|---|
| <1 | Not perceived as different by the human eye |
| 1-2 | Perceptible upon close observation (JND) |
| 2-10 | Perceptible at a glance |
| 11-49 | Colours are more similar than opposite |
| 49-100 | Colours are more opposite than similar |

### Example 1. Synthesis of Porphyrin Hosts

### Synthesis of 5,10,15,20-tetrakis(2-nitrophenyl)porphyrin, H₂TNPP

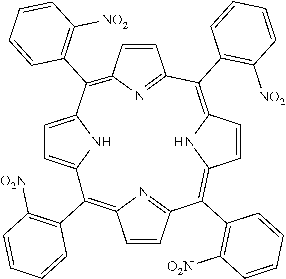 [SMILES: O=[N+]([O-])c1ccccc1-c1c2nc(c(-c3ccccc3[N+](=O)[O-])c3ccc([nH]3)c(-c3ccccc3[N+](=O)[O-])c3nc(c(-c4ccccc4[N+](=O)[O-])c4ccc1[nH]4)C=C3)C=C2]

This synthesis was performed according to the procedure of Sorrell et al. (2007). 2-Nitrobenzaldehyde (6.43 g, 0.043 mol) was dissolved in glacial acetic acid (120 mL) in a 2 L, three necked round bottom flask. The solution was heated to boiling with vigorous stirring. Pyrrole (2.45 g, 0.037 mol) was added dropwise. The dark solution was stirred at reflux for 30 minutes before cooling to 35° C. with the dropwise addition of chloroform (18 mL). The solution was left overnight. The crystal product was collected by vacuum filtration and washed with chloroform (5×20 mL). The product was dried in an oven at 100° C. overnight. Yield: 1.80 g (6%). ¹H NMR ((CD₃)₂SO) δ8.73 (s), 8.63-8.12 (m, broad), 5.75 (s), 3.86 (s), -2.75 (s).

### Synthesis of 5,10,15,20-tetrakis(2-aminophenyl)porphyrin, H₂TAPP

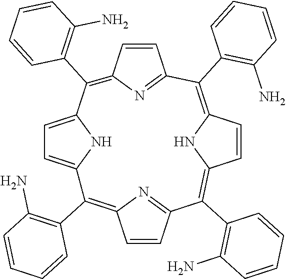 [SMILES: Nc1ccccc1-c1c2nc(c(-c3ccccc3N)c3ccc([nH]3)c(-c3ccccc3N)c3nc(c(-c4ccccc4N)c4ccc1[nH]4)C=C3)C=C2]

This synthesis was performed according to the procedure of Sorrell et al. (2007). 5,10,15,20-Tetrakis(2-nitrophenyl)porphyrin (0.90 g, 0.001 mol) was combined with concentrated hydrochloric acid (45 mL) and combined with tin(II) chloride hydrate (3.75 g, 0.016 mol) in concentrated hydrochloric acid (3.75 mL) and stirred at room temperature for 90 minutes. The solution was then warmed to 65° C. over 10 minutes. The temperature was maintained at 65-70° C. for 25 minutes with vigorous stirring. The solution was moved to an ice bath to cool to room temperature. Concentrated ammonium hydroxide (45 mL) was added to neutralise (pH>10). The solution was stirred overnight with chloroform (75 mL). The organic layer was collected, and the aqueous layer was combined with additional water (115 mL). The aqueous layer was extracted with chloroform (3×12 mL). The organic phases were combined and washed with dilute ammonium hydroxide solution (75 mL). The aqueous solution was then extracted with chloroform (2×5 mL). The combined organic phases were filtered and the filtrate was concentrated to ˜30% volume using rotary evaporation. The solution was then combined with ethanol (12 mL) and ammonia solution (0.75 mL). The solution was left to slowly evaporate under a stream of air, and a black microcrystalline precipitate was formed. The solid was collected by filtration and dried in an oven at 100° C. overnight. Yield: 0.288 g (39%). ¹H NMR ((CD₃)₂SO) δ8.79 (s, 8H), 7.68 (m, 4H). 7.52 (m, 2H), 7.14 (m, 4H), 7.00 (m, 4H), −2.68 (s, 2H). ¹³C NMR ((CD₃)₂SO) δ148.7, 134.9, 129.9. 125.6, 116.7, 115.3, 79.6. 56.5, 19.0.

### Synthesis of α,α,α,α-5,10,15,20-tetrakis(2-aminophenyl) porphyrin, H₂TAPP (7)

This procedure was performed according to the method of Lindsey (2002). Benzene (85 mL) and silica gel (36 g) were combined in a three neck round bottom flask connected to a nitrogen inlet and a reflux condenser. The solution was stirred and warmed to 80° C. in an oil bath for two hours to saturate the atmosphere. 5,10,15,20-tetrakis(2-aminophenyl)porphyrin (1.00 g, 1.48 mmol) was added. The mixture was stirred for 20 hours. The dark slurry was cooled to room temperature and poured onto a wide glass frit. A solution of ether and benzene (1:1) was added until a red colour change was visible in the filtrate (˜300 mL). A solution of acetone and ether (1:1) was then added until the red colour was no longer visible in the filtrate (˜200 mL). The like fractions were collected by comparison of TLCs. The solvent was removed using rotary evaporation to yield a dark purple solid. Yield: 0.435 g (58%). ¹H NMR (CDCl₃) δ8.79 (s, 8H), 7.69 (d, 4H, J=5.94 Hz). 7.52 (t, 4H, J=7.80 Hz), 7.14 (d, 4H, J=8.34 Hz), 7.00 (t, 4H, J=7.38 Hz). ¹³C NMR (CDCl₃) δ148.6, 134.9, 125.6, 116.7, 116.1, 115.3, 69.0.

### Synthesis of α,α,α,α-5,10,15,20-tetrakis(2-(pentadeccoborooctanoylamino)phenyl) porphyrin (9)

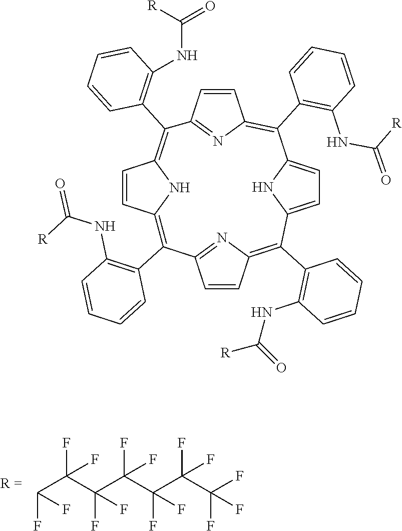

This procedure was performed according to the method of Kölmel et al (2013). α,α,α,α-5,10,15,20-Tetrakis(2-aminophenyl)porphyrin (1 eq) and anhydrous pyridine (1.2 eq) were combined in dry dichloromethane and stirred at room temperature for an hour. A solution of pentadecafluorooctanoyl chloride (4.2 eq) in dry dichloromethane was added dropwise. The mixture was stirred overnight at room temperature in the absence of light. Dichloromethane was added. The mixture was washed with water, aqueous HCl (0.1 M) and saturated aqueous NaHCO₃. The organic phase was dried using Na₂SO₄ and the solvent was removed using rotary evaporation to yield a dark crystalline purple material. Crystals suitable for x-ray diffraction were grown by slow evaporation from hexane. (Yield ˜70%). ¹H NMR (CDCl₃) δ8.72 (s, 4H), 8.58 (d, J=8.46 Hz), 7.87 (m, 4H), 7.71 (s, 2H), 7.56 (t, J=7.44 Hz), 1.44 (s, 8H), −2.75 (s, br, 1H). ¹³C NMR (CDCl₃) δ145.8, 136.6, 132.0, 129.2, 126.9, 125.6, 110.1, 25.6, 7.01. ¹⁹F NMR (CDCl₃) δ−81.3, −120.9, −122.3, −122.6, −123.6, −126.7. MS (ITMS): m/z for C₇₆H₃₀F₆₀N₈O₄ 2671.1091.

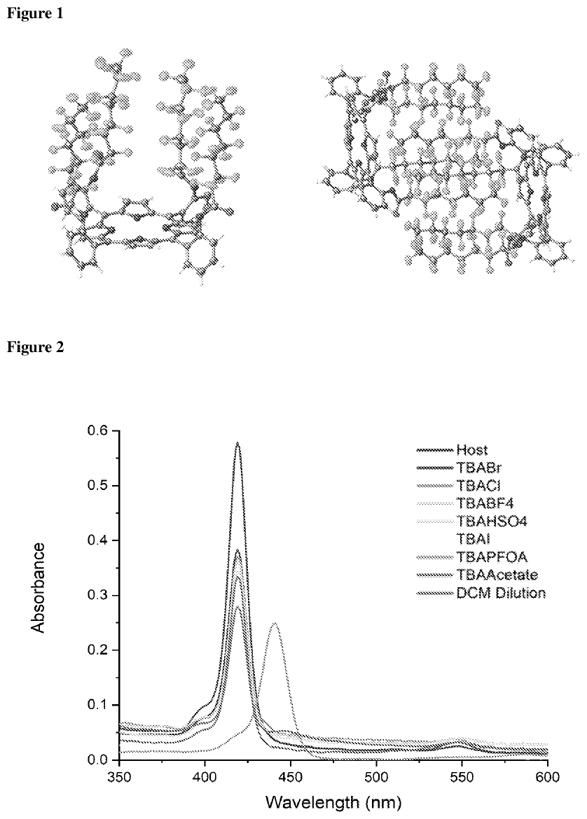

An X-ray crystal structure of the product, α,α,α,α-5,10,15,20-tetrakis(2-(pentadecafluorooctanoylamino)phenyl)porphyrin, demonstrated the potential for the cavity to bind perfluorinated alkanes as it crystallises as a dimer with one of the perfluorinated chains of each molecule inside the neighbouring cavity (FIG. 1).

### Synthesis of 5,10,15,20-tetra-(2-ureaphenyl-pentadecafluoro-octanoyl) porphyrin (10)

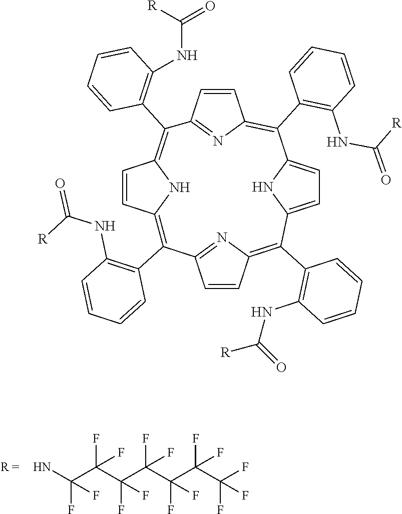

This synthesis was performed using the method of Usachova et al (2010). Perfluorooctanohydroxamic acid (0.28 g, 0.61 mmol) was dissolved in acetonitrile (3 mL) with CDI (0.11 g, 0.72 mmol) and stirred at room temperature for thirty minutes. The solution was then heated to 60° C. and stirred for two hours. H₂TAPP (0.14 g, 0.06 mmol) was added and the solution was cooled to room temperature and stirred overnight. The solution was diluted with ethyl acetate and washed twice with saturated aqueous NH₄Cl solution. The organic phase was then washed with deionised water, and then brine. The organic phase was dried over Na₂SO₄. The solution was filtered and concentrated using rotary evaporation. (Yield ˜56%). ¹H NMR (CDCl₃) δ8.79 (m, 8H), 8.66 (m, 6H), 8.57 (m, 2H), 7.92 (m, 8H), 7.63 (m, 8H), −2.68 (s, br, 2H). ¹⁹F NMR (CDCl₃) −126.50 (2F), −123.26-−120.24 (12F), −81.05 (3F).

### Synthesis of α,α,α,α-5,10,15,20-tetra(2-(heptafluorobutanoylamino)phenyl) porphyrin (11)

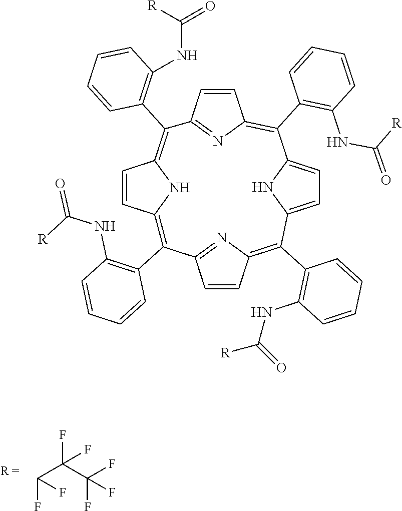

α,α,α,α-5,10,15,20-Tetrakis(2-aminophenyl)porphyrin (1 eq) and pyridine (8 eq) were combined in dry dichloromethane (10 mL) and stirred at room temperature for an hour. A solution of perfluorobutyryl chloride (4 eq) in dry dichloromethane (5 mL) was added dropwise. The mixture was stirred overnight at room temperature. Dichloromethane was added. The mixture was washed with water, aqueous HCl (10%) and saturated aqueous NaHCO₃. The organic phase was dried using Na₂SO₄ and the solvent was removed using rotary evaporation to yield a dark crystalline purple material. (Yield 74%). Crystals suitable for x-ray diffraction were grown by slow evaporation from dichloromethane. Crystals were dried under high vacuum. ¹H NMR (CDCl₃) δ8.62 (s, 8H), 7.98 (d, 1H, J=12 Hz), 7.92 (t, 1H, J=12 Hz), 7.76 (t, 1H, J=6 Hz), 7.64 (t, 1H, J=12 Hz), 7.36 (t, 1H, J=6 Hz), −2.69 (s, br, 1H). ¹³C NMR (CDCl₃) δ148.8, 137.1, 135.8, 135.1, 132.4, 130.5, 125.3, 124.1, 121.9, 113.9. ¹⁹F NMR (CDCl₃) δ−81.9, −122.1, −128.4.

### Synthesis of α,α,α,α-5,10,15,20-tetrakis(2-(tricosafluorododecanoylamino)phenyl) porphyrin (12)

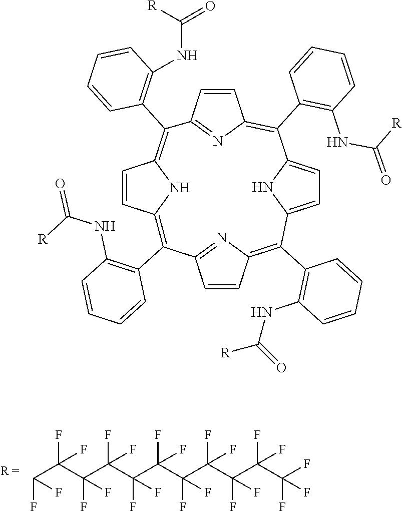

Tricosafluorododecanoic acid (4 eq) was combined with thionyl chloride (10 eq) under an atmosphere of nitrogen and heated at reflux overnight. The solvent was removed under vacuum to yield white crystals, and dry toluene added. The solvent was removed by vacuum. Dry dichloromethane was added, and the solution stirred at room temperature. α,α,α,α-5,10,15,20-Tetrakis(2-aminophenyl)porphyrin (1 eq) and pyridine (1.2 eq) were combined in dry dichloromethane and stirred at room temperature for an hour. The solution of tricosafluorododecanoyl chloride (1.2 eq) in dry dichloromethane was added dropwise. The mixture was stirred overnight at room temperature. Dichloromethane was added. The mixture was washed with water, aqueous HCl (10%) and saturated aqueous NaHCO₃. The organic phase was dried using Na₂SO₄ and the solvent was removed using rotary evaporation to yield a dark crystalline purple material (Yield ˜48%). ¹H NMR (CDCl₃) δ8.62 (s, 1H), 7.86 (m, 1H), 7.60 (m, 1H), 7.31 (t, 1H, J=7.2 Hz), 7.17 (m, 1H), 7.11 (m, 1H), −2.67 (s, br, 1H). ¹³C NMR (CDCl₃) δ145.83, 133.78, 128.69, 128.03, 1287.22, 125.87, 124.29, 116.55, 116.52, 114.81, 114.23 ¹⁹F NMR (CDCl₃) −126.08 (3F), −122.60-−122.58 (14F), −117.17 (3F), −80.75 (4F).

### Synthesis of 5,15-bis-(2-amidophenyl-pentadecafluoro-octanoyl) porphyrin (13)

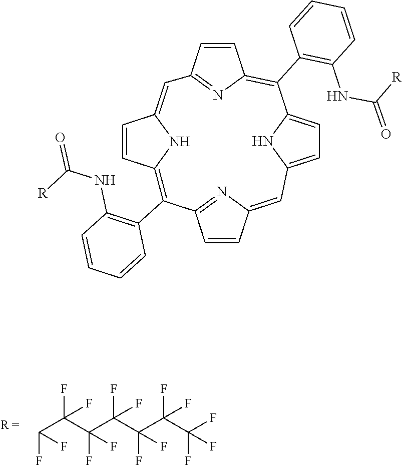

5,15-Bis-(2-aminophenyl)porphyrin (1 equiv.) (0.02 g, 0.0004 mol) was dissolved in dry dichloromethane (5 mL) in the absence of light. Perfluorooctanoyl chloride (2 equiv.) (0.03 g, 0.0008 mol) was added to a solution of pyridine (8 equiv.) (0.25 g, 0.0032 mol) in dry dichloromethane (10 mL). The solution of 5,15-bis-(2-aminophenyl)porphyrin was then added via syringe. The combined solution was stirred in the dark overnight. The organic phase was washed with water (20 mL), dilute aqueous HCl (20 mL) and saturated NaHCO₃ solution. The organic phase was dried over Na₂SO₄ and filtered through a glass frit. The organic phase is dried under a stream of air. Yield: 9%. ¹H NMR (CDCl₃) δ10.43 (s, 2H), 9.42 (s, 4H), 8.85 (s, 4H), 8.23 (dd, J=7.44, 32.22 Hz, 2H), 8.13 (m, 2H), 7.91 (t, J=8.10 Hz, 2H), 7.76 (m, 2H).

### Synthesis of (5,10,15,20-tetrakis[2-(4-perfluorooctyl-1H-1,2,3-triazolyl)phenyl]porphyrinato)zinc(II) (14)

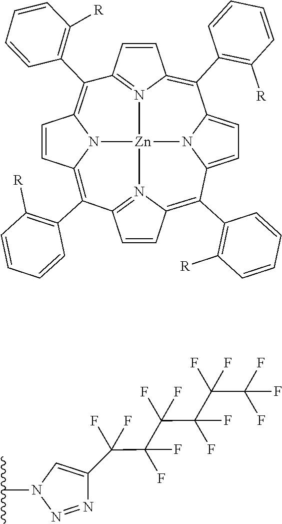

This synthesis was performed according to a modified method by Lee et al (2011). CuSO₄.5H₂O (0.012 g, 0.05 mmol) and sodium ascorbate (0.01 g, 0.05 mmol) were added to a mixture of zinc [5,10,15,20-tetrakis[2-(azidomethyl)phenyl]porphyrin (0.01 g, 0.01 mmol) and 3,3,4,4,5,5,6,6,7,7,8,8,9,9,10,10,10-heptadecafluorodec-1-yne (0.025 g, 0.05 mmol, MW=443.98) in THF/H₂O (1:1) (5 mL). The reaction mixture was stirred for 12 hours at 50° C. The solution was cooled to room temperature and extracted with dichloromethane (3×10 mL), the organic layer was separated, dried over Na₂SO₄, and filtered. After evaporation of the solvent under reduced pressure, the residue was purified using column chromatography with 70% ethyl acetate/dichloromethane, and the second fraction was collected and evaporated to dryness. The residue was recrystallised from dichloromethane/hexane to produce a purple solid. (Yield ˜42%) ¹H NMR (CDCl₃) δ8.79 (s, 4H), 6.85 (t, 8H, J=13.56 Hz), 6.49 (d, 8H, J=15.66 Hz), 5.90 (m, 12H), 5.29 (m, 2H), 4.36 (t, 8H, J=7.02 Hz). ¹³C NMR (CDCl₃) δ116.16, 110.70, 97.18, 68.72, 68.49, 65.01, 30.27, 29.69, 29.63, 29.32. ¹⁹F NMR (CDCl₃) δ−80.74 (2F), 121.85-122.66 (12F), 126.06 (3F).

### Synthesis of (5,10,15,20-tetrakis[2-(4-perfluorooctyl-1H-1,2,3-triazolyl)phenyl]porphyrin (15)

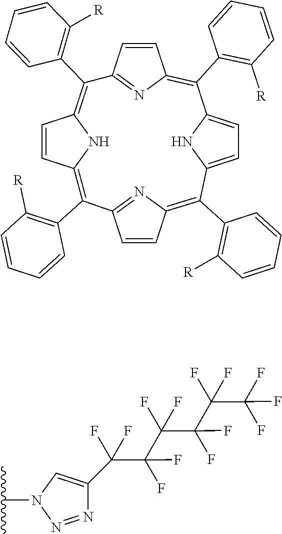

HCl (37%; 2.5 mL) was added to an ethyl acetate (10.0 mL) solution of 5,10,15,20-tetrakis [2-(4-perfluorooctyl-1H-1,2,3-triazolyl)phenyl]porphyrinato)zinc(II) (48.0 mg, 0.018 mmol). The resulting solution was stirred for 3 hours at room temperature. Then water (10 mL) was added, and the organic phase was washed with water until neutral (pH 7). The resulting solution was dried over Na₂SO₄ and filtered. The solvent was evaporated to dryness under vacuum to yield a purple solid.

### Synthesis of tetra butyl ammonium pentadecafluoro-octanoate

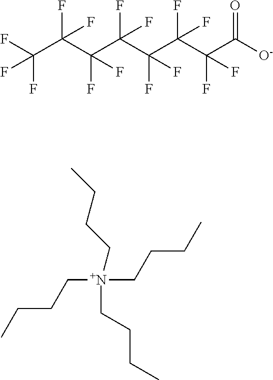 [SMILES: O=C([O-])C(F)(F)C(F)(F)C(F)(F)C(F)(F)C(F)(F)C(F)(F)C(F)(F)F ; CCCC[N+](CCCC)(CCCC)CCCC]

This synthesis was performed according to the procedure of Bales et al (2004). Perfluorooctanoic acid (0.200 g, 0.48 mmol) was dissolved in warm deionised water with vigorous stirring. The solution was allowed to cool to room temperature before tertbutylammonium hydroxide was added dropwise. The crystalline precipitate was collected by filtration and washed with additional cold water. ¹H NMR (CDCl₃) δ3.32-3.29 (m, 8H), 1.67 (m, 8H), 1.46, (m, 8H), 1.02 (t, 12H, J=7.32 Hz). ¹³C NMR (CDCl₃) δ165.1, 119.0, 110.0, 108.2, 106.2, 60.1, 24.8, 18.9, 14.7.

### Synthesis of tetra butyl ammonium acetate

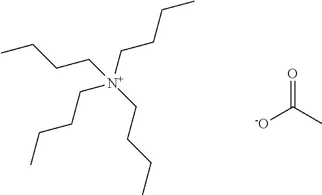

This synthesis was performed according to the procedure of Xu et al (2011). Potassium acetate (0.88 g, 9.0 mmol) and tetrabutylammonium chloride (2.50 g, 9.0 mmol) were combined in methanol under an atmosphere of nitrogen. The solution was stirred at room temperature overnight. The precipitated solid was removed by filtration. The filtrate was reduced using rotary evaporation and the resulting solid was dried in an oven for 24 hours. ¹H NMR (D₂O) δ3.22-3.17 (m, 8H), 1.91 (s, 3H), 1.65 (m, 8H), 1.41-1.31 (m, 8H), 0.95 (t, 12H, J=7.44 Hz). ¹³C NMR (D₂O) δ63.2, 61.5, 56.5, 32.6, 31.1, 30.0, 18.9.

### Example 2. UV-Vis Binding Studies

Preliminary binding studies were undertaken by combining an amide linked porphyrin host molecule (9) and tetrabutylammonium perfluorooctanoate (TBAPFO) in dichloromethane.

The interactions of amide linked porphyrin host molecule (9) with TBAPFO were investigated with ¹H NMR spectroscopy but self-aggregation effects at the higher concentration of host molecule (9) prevented full characterisation of intermolecular binding interactions. The self-aggregation behaviours were observed by analysing a series of serial dilutions with UV-Vis spectroscopy. The shift in the Soret and Q bands remained constant below 10⁻⁶ M concentrations. Accordingly, binding studies were performed at or below the 10⁻⁶ M concentrations where aggregation did not have an impact.

The red-brown solution of host (9) changed colour to green upon the addition of solid TBAPFO. The colour change of the host solution could also be observed for a biphasic mixture of host solution in dichloromethane (0.0124 mmol, 5 mL) and TBAPFO (3 ppm, 0.0058 mmol, 500 mL) in water.

The binding interaction was probed by combining the host (9) with non-fluorinated and neutral fluorinated guest molecules. Solutions of host (9) in dichloromethane were combined with 10 molar equivalents of tetrafluorobenzene and nonanoic acid. Solutions were analysed using UV-Vis spectroscopy (immediately and 24 hours later) to check for any indication of binding. In both instances, there was no colorimetric response. This suggested that the binding interaction observed for PFOA and host (9) is due to a combination of both the anionic carboxylic acid and the fluorination.

The selectivity of the host (9) for anions was investigated visually and spectroscopically. The TBA salts of anions commonly found in significant concentrations in water samples were added in 10 molar equivalents to solutions of host to give preliminary indications of host−guest interactions prior to more extensive investigation. Although not an accurate proxy of real-world matrix concentrations, it was thought that the binding of any anions would indicate any affinity for other anions. UV-Vis spectroscopic analysis showed no significant change after the addition of Cl⁻, Br⁻, I⁻, HSO₄⁻, CH₃COO⁻, BF₄⁻, but a shift in the Q band (˜32 nm) upon addition of the PFOA⁻ (FIG. 2).

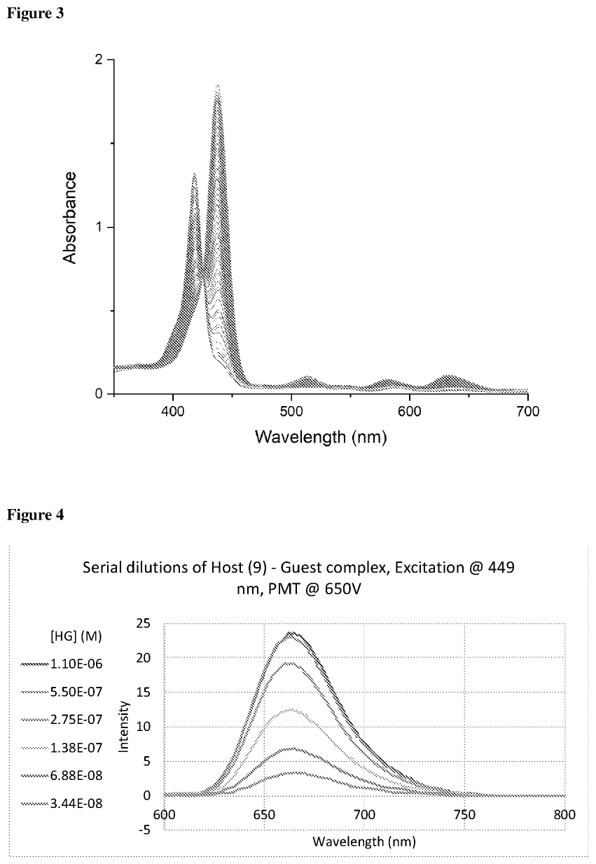

UV-Vis spectroscopy binding studies were used to determine the association constant for the host (9)−PFOA complex. A UV-Visible host−guest addition titration of host (9) (1.01×10⁻⁶ M) in dichloromethane with aliquots containing 0.1 molar equivalents of TBAPFO showed the λₘₐₓ of (9) undergoes a red shift from 417 to 444 nm upon coordination of the PFOA anion (FIG. 3). The isosbestic point indicates the formation of a host−guest complex that does not transition through an intermediary state. Using BindFit isotherm simulators, the association constant (Kₐ) for the formation of the host−guest complex was determined to be in the order of ˜2.80×10⁶ M⁻¹ from both Nelder-Mead and L-BFGS-B methods (Thordarson, 2011). Data fitting was also attempted applying 2:1 and 1:2 binding models, with the output suggesting that a 1:1 system was the most reasonable fit (based on calculated error).

The limit of detection of host (9) with PFOA was probed using fluorescence spectrophotometry. In these experiments, solutions of the 1:1 host−guest complex (host (9)+PFOA) in dichloromethane were analysed by measuring the characteristic host−guest peak after excitation. The solutions were sequentially diluted until the signal was no longer discernible above instrument noise (FIG. 4).

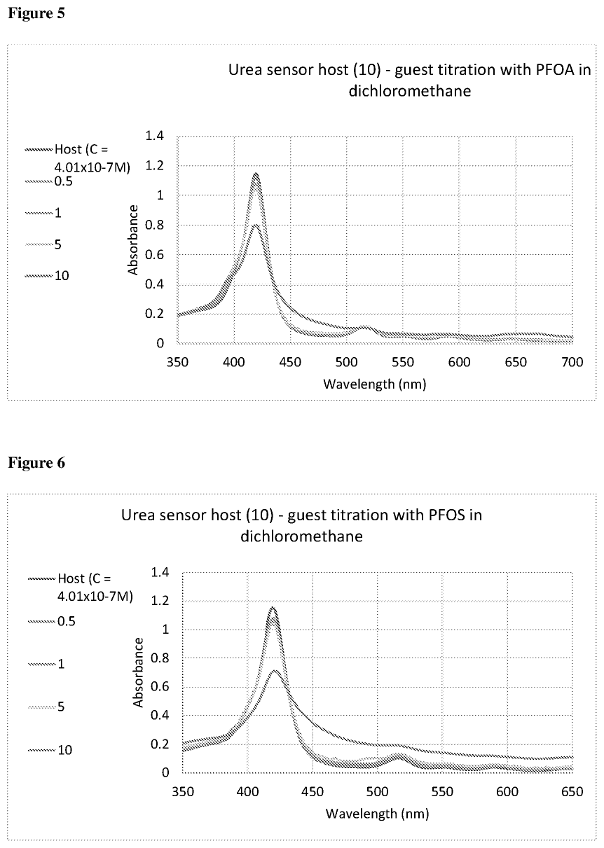

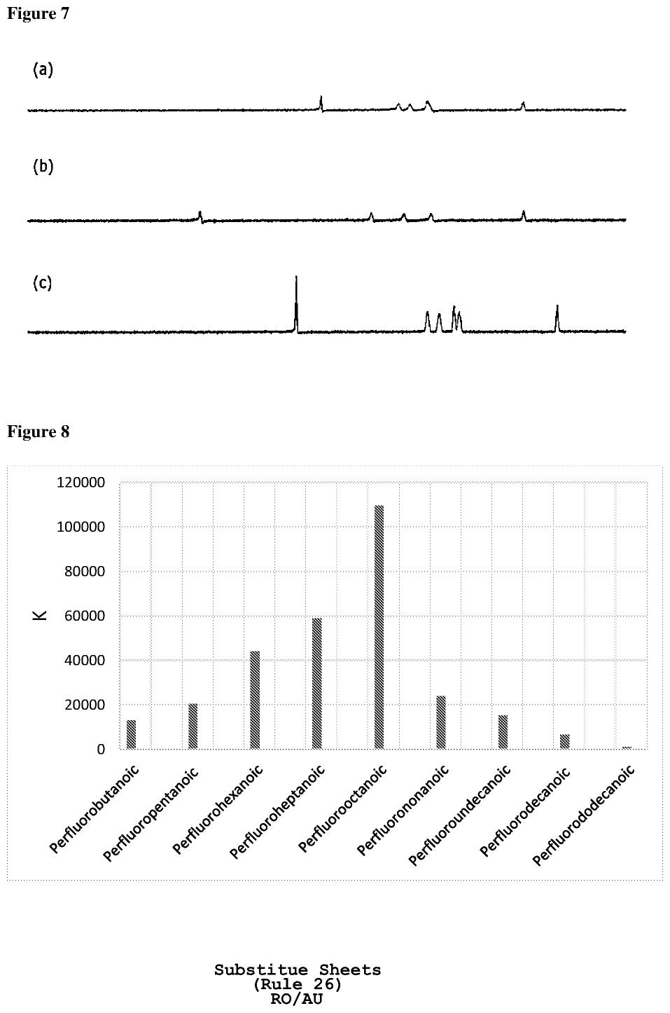

Preliminary binding studies of a urea linked porphyrin host molecule (10) showed that the addition of PFOA (FIG. 5) and PFOS (FIG. 6) caused a discernible colour change by UV-Vis spectrophotometric analysis. However, these colorimetric changes were weaker than that observed with the amide equivalent, likely due to the formation of an insoluble precipitate upon binding. Binding of the urea linked porphyrin host with PFOA and PFOS was also confirmed by ¹⁹F NMR spectra (FIG. 7).

The amide linked porphyrin host (9) was also combined and analysed with a range of different length PFAS (Table 2) and analysed by UV-Vis spectroscopy. The UV-Vis spectrophotometric analysis suggested a relationship between the PFCA chain length and its size matching to the host molecule cavity.

A host solution of host (9) in dichloromethane was monitored using UV-Vis analysis as 0.5-10 equivalents of each PFAS was added. The binding data was collected in triplicate for both the TBA salt and acid equivalents of the PFAS due to differences in associated binding strengths. The data was processed using Bindfit isotherm simulators using both Nelder-Mead and L-BFGS-B methods (Thordarson, 2011). The averaged association constant (Kₐ) for the formation of the host−guest complex of each PFAS is shown in Table 2 and FIG. 8.

TABLE 2
Averaged association constants (Kₐ) for
perfluorocarboxylic acid (PFCA) guests

| Formula | PFAS | Acronym | K value | Error % |
|---|---|---|---|---|
| C₃F₇COOH | Perfluorobutanoic acid | PFBA | 12999 | 0.93 |
| C₄F₉COOH | Perfluoropentanoic acid | PFPeA | 20459 | 0.85 |
| C₅F₁₁COOH | Perfluorohexanoic acid | PFHxA | 392106 | 7.5 |
| C₆F₁₃COOH | Perfluoroheptanoic acid | PFHpA | 58920 | 3.7 |
| C₇F₁₅COOH | Perfluorooctanoic acid | PFOA | 109574 | 5.3 |
| C₈F₁₇COOH | Perfluorononanoic acid | PFNA | 23968 | 2 |
| C₉F₁₉COOH | Perfluoroundecanoic acid | PFDA | 15272 | 0.92 |
| C₁₀F₂₁COOH | Perfluorodecanoic acid | PFUnDA | 6585 | 0.97 |
| C₁₁F₂₃COOH | Perfluorododecanoic acid | PFDoDA | 1010 | 0.26 |

Addition of all nine PFAS trialled produced a visual colour change in the host (9) solutions, but the trends in the associated binding constants suggested a preference in the size similarity of the PFAS and the receptor fluorinated chain length. The size complimentary trend was probed by modifying the length of the fluorinated “arms” of the receptor.

A “short” chain host (11) having a butyl-fluorinated chain and a “long” chain receptor (12) having a dodecyl-fluorinated chain were also combined with PFOA and their binding constants compared (Table 3).

TABLE 3
Association constants and error for short, medium
and long chain sensor molecules with PFOA

| Host | K (M⁻¹) | K error (%) | Log K |
|---|---|---|---|
| Short (11) | 1.19 × 10⁶ | 5 | 6.07 ± 0.7 |
| Medium (9) | 1.77 × 10⁶ | 7 | 6.25 ± 0.8 |
| Long (12) | 1.67 × 10⁶ | 18 | 6.22 ± 1.3 |

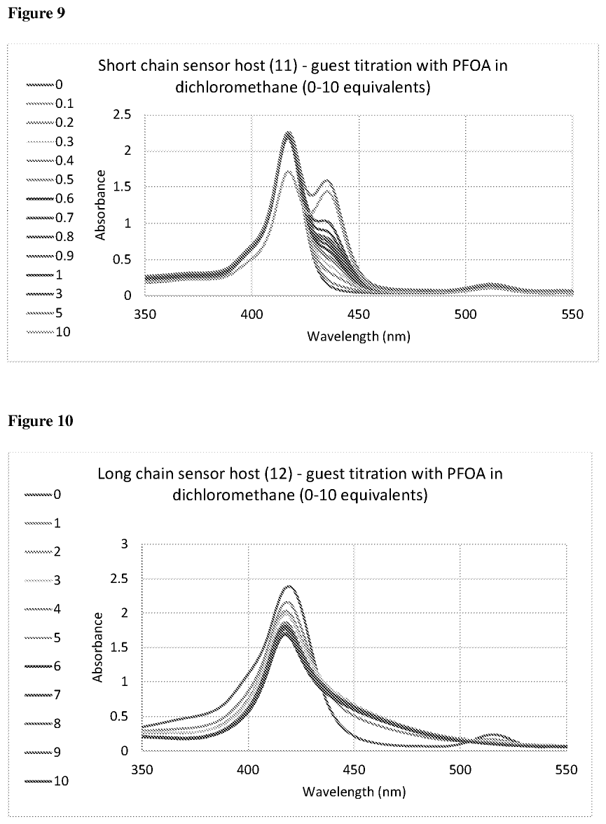

A preliminary equivalent host−guest titration of long chain host (12) with PFOA showed a lower association constant than that of short chain host (11), but also a change in binding behaviour and aggregation habits (FIGS. 9 and 10). These were observed from UV-Vis analysis of serial dilutions, and comparison of association constants when modelled for 1:1, 1:2 and aggregation systems. The larger association constants were accompanied by larger associated error.

The “medium” chain host (9) showed a preferential size match (i.e., demonstrated higher binding affinity) for PFOA. Accordingly, the “short” chain host (11) was tested to determine whether it would be a better host (i.e., have a stronger binding affinity) for a PFAS such as perfluorobutanoic acid or perfluoropentanoic acid. Similarly, the “long” chain host (12) was tested to determine whether it would be a better host for the long chain PFAS such as perfluoroundecanoic acid. Each sensor was screened with the PFCAs ranging in carbon chain length from 4-12. A solution of host (2.01×10⁻⁹ M) in dichloromethane was combined with 10 molar equivalents of each PFAS. The solutions were observed visually and analysed using UV-Vis spectrophotometry.

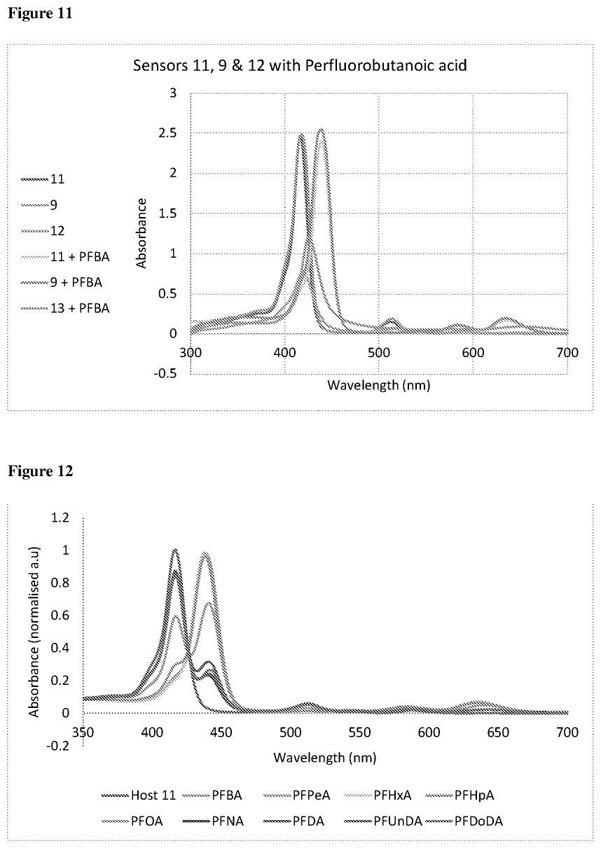

Visually, there was a difference in the intensity of the green colour change with increasing PFAS chain length. The UV-Vis analysis of each solution highlighted the preferential binding habits of each sensor (FIGS. 9 and 10). When PFAS was bound to the sensor there was a distinct shift in the Soret band as shown in FIG. 11.

### Example 3. RGB Detection of Perfluorocarboxylic Acids

### PFCA Guest Response Screening

Experiments were performed at concentrations that could be monitored by the naked eye for practical sensing of PFAS in the field. Complexes of hosts (9), (11) and (12) were formed with the nine different perfluorocarboxylic acids (PFCAs) shown in Table 2 above and the colours of each host−PFCA complex were photographed in a lightbox and analysed using ImageJ software RGB analysis. Pixels of the solution were chosen at random to provide an RGB value. Multiple RGB values were recorded for each host−PFCA solution, and an average colour can be produced. From those values an RGB parameter was used to give a response value for modelling (Tahir et al., 2016).

After preliminary screenings, UV-Vis spectra were collected before and after the addition of 1 molar equivalent of each PFCA for each host, and the degree of complexation was noted by shifts in the Soret bands and formation of a peak at ca. 650 nm. To eliminate dilution effects, PFCA guest solutions were prepared in the equivalent concentration of host. If there was a notable host−guest interaction for a particular PFCA, the host−guest binding was investigated with more rigorous UV-Vis experiments, binding studies, and further colour analyses. Throughout these investigations, PFOA was considered the “average” PFCA both because of its position in the range of PFCA sizes and because its prevalence is often an indication of broader PFAS contamination. In any experiment where binding was further investigated for a particular PFCA, it was typically replicated with PFOA for comparison.

### PFCA Guest Response−Short chain Host (11)

Host (11) provided a strong colorimetric response to all nine PFCAs, as evidenced by UV-vis spectroscopy where a shift of the λₘₐₓ was observed upon complexation. The Soret band of the host molecule (11) was observed at ca. 416 nm whilst the Soret band of the host−PFCA complex was observed at ca. 444 nm (FIG. 12). The addition of PFBA resulted in high conversion to the host−guest complex, while the addition of PFDoDA indicated the presence of significant quantities of both host and a host−guest complex. Table 4 below shows the RGB data from ImageJ analysis for host (11), comparison of the change in red and green values in solution in response to PFCA addition, and the ΔE host−PFCA for each complex. Although all the PFCA containing samples were visibly green, the green intensity was observed to decrease with increasing length of PFCA, as evidenced by the change in the relative RGB values (Table 4).

TABLE 4
RGB data for host (11) with PFCAs

| | PFBA | PFPeA | PFHxA | PFHpA | PFOA | PFNA | PFDA | PFUnDA | PFDoDA | Host |
|---|---|---|---|---|---|---|---|---|---|---|
| Av(Red) | 158 | 156 | 157 | 159 | 161 | 169 | 167 | 172 | 170 | 194 |
| Av(Green) | 178 | 177 | 179 | 178 | 172 | 169 | 165 | 169 | 165 | 177 |
| Av(Blue) | 78 | 78 | 83 | 84 | 82 | 106 | 102 | 98 | 101 | 162 |
| G − R | 20 | 21 | 22 | 19 | 11 | 0 | −2 | −3 | −5 | −17 |
| Av(RGB) | 138 | 137 | 140 | 140 | 138 | 148 | 145 | 146 | 145 | 178 |
| ΔE_{Host-PFCA} | 45.9 | 45.8 | 44.4 | 43.2 | 40.6 | 26.8 | 27.0 | 30.5 | 27.2 | |

### PFCA Guest Response−Medium Chain Host (9)

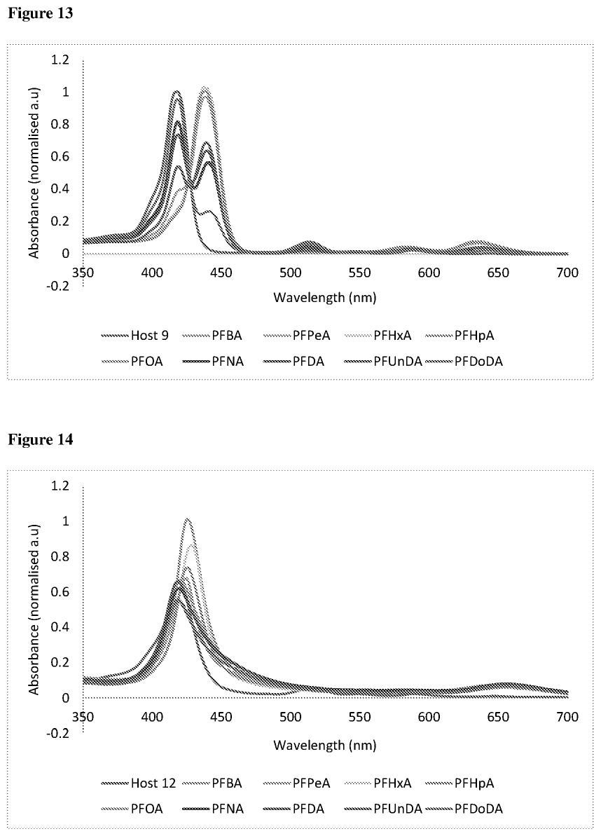

A colorimetric response was visible for all tested PFCAs when combined with host (9) in dichloromethane. The intensity and brightness of the green colour observed was stronger for the short to medium chain length PFCAs. The formation of the host−guest Soret bands indicated binding across the entire range of PFCA guests (FIG. 13), showing that host (9), like host (11), is a strong receptor for all the tested PFCAs. The variability (green and red difference) between colour values decreases as the length of the PFCA increases, detectable visually in solution primarily by the green hue or intensity. Table 5 below shows the RGB data from ImageJ analysis for host (9), comparison of the change in red and green values in solution in response to PFCA addition, and the ΔE_{Host-PFCA} for each complex.

TABLE 5
RGB data for host (9) with PFCAs

| | PFBA | PFPeA | PFHxA | PFHpA | PFOA | PFNA | PFDA | PFUnDA | PFDoDA | Host |
|---|---|---|---|---|---|---|---|---|---|---|
| Av(Red) | 155 | 153 | 153 | 151 | 163 | 164 | 160 | 161 | 165 | 184 |
| Av(Green) | 179 | 177 | 179 | 179 | 175 | 173 | 174 | 168 | 162 | 167 |
| Av(Blue) | 78 | 76 | 76 | 77 | 86 | 85 | 82 | 80 | 106 | 151 |
| G − R | 24 | 24 | 26 | 28 | 12 | 10 | 14 | 7 | −3 | −17 |
| Av(RGB) | 137 | 135 | 136 | 136 | 141 | 141 | 139 | 137 | 144 | 167 |
| ΔE_{Host-PFCA} | 46.1 | 46.2 | 47.3 | 47.2 | 39.3 | 38.5 | 39.6 | 38.5 | 22.4 | |

To highlight any potential binding preferences across the PFCAs from the host molecules, triplicates of host−guest addition titrations were performed using host (9) and the TBA salts of the nine different deprotonated PFCAs. Host (9) was used because its response to the PFCAs could be most consistently modelled. The association constants were within the same order of magnitude across the range of PFCA guests (Table 6), but the colorimetric response varied with chain length; the RGB colour analysis showed the intensity and change in average colour values were significant across the range of PFCAs (Table 5).

TABLE 6
Association constants (logK) for host (9) with PFCAs

| PFCA | PFBA | PFPeA | PFHxA | PFHpA | PFOA | PFNA | PFDA | PFUnDA | PFDoDA |
|---|---|---|---|---|---|---|---|---|---|
| LogK | 5.3 ± | 5.3 ± | 5.7 ± | 6.1 ± | 6.2 ± | 5.3 ± | 4.9 ± | 4.0 ± | 4.1 ± |
| | 1.2 | 1.3 | 1.1 | 1.5 | 0.9 | 0.9 | 0.6 | 0.2 | 0.1 |

### PFCA Guest Response−Long Chain Host (12)

Host (12) also provided a colorimetric response to all nine PFCAs. The Soret band of host (12) is less pronounced than that of (9) and (11), but the formation of a peak at ca. 650 nm upon binding is clearly distinguishable in all three host molecules. For that reason, absorbance values at the characteristic host−guest peak at ca. 650 nm are useful to show the response to each PFCA (Table 7), as the shifts in the Soret band for host (12) show less of a diagnostic switch (FIG. 14). Table 7 below shows the absorbance at the characteristic host−PFCA complex peak observed at 650 nm for the three host molecules. The nine PFCAs show a significantly higher absorbance than the host (9), with shorter PFCAs giving the strongest response. Host (11) showed a significant decrease between PFOA and PFNA. Host (12) showed the most consistent response across all nine PFCAs.

TABLE 7
Absorbance at the characteristic host - PFCA complex peak
observed at 650 nm for host molecules (9), (11) and (12)
Absorbance @ 650 nm

| | Host (11) | Host (9) | Host (12) |
|---|---|---|---|
| PFBA | 0.113 | 0.04939 | 0.0906 |
| PFPeA | 0.1125 | 0.04302 | 0.0692 |
| PFHxA | 0.0967 | 0.04964 | 0.0728 |
| PFHpA | 0.1009 | 0.04879 | 0.0737 |
| PFOA | 0.0977 | 0.02885 | 0.0913 |
| PFNA | 0.0473 | 0.02986 | 0.0953 |
| PFDA | 0.0469 | 0.03023 | 0.0918 |
| PFUnDA | 0.0475 | 0.02958 | 0.0808 |
| PFDoDA | 0.0519 | 0.01719 | 0.0769 |
| Host/control | 0.0019 | 0.00113 | 0.132 |

Unlike hosts (9) and (11), there was no obvious size-based binding trend or preference impacting the colours for host (12) with the tested PFCAs. Without wishing to be bound by theory, the present inventors have postulated that this may because host (12) has a broadening of absorption bands upon complexation of PFCAs as opposed to the shifts in absorption peaks observed in the spectra for hosts (9) and (11). The ImageJ analysis also showed there was less variability in the RGB values and perceived colour across the host−PFCA complexes (Table 8).

TABLE 8
RGB data for host (12) with PFCAs

| | PFBA | PFPeA | PFHxA | PFHpA | PFOA | PFNA | PFDA | PFUnDA | PFDoDA | Host |
|---|---|---|---|---|---|---|---|---|---|---|
| Av(Red) | 177 | 169 | 167 | 166 | 170 | 166 | 170 | 168 | 181 | 193 |
| Av(Green) | 182 | 171 | 174 | 174 | 176 | 173 | 174 | 169 | 177 | 184 |
| Av(Blue) | 110 | 126 | 112 | 115 | 104 | 101 | 101 | 104 | 111 | 151 |
| G − R | 5 | 2 | 6 | 8 | 6 | 8 | 3 | 1 | −4 | −9 |
| Av(RGB) | 156 | 155 | 151 | 152 | 150 | 147 | 148 | 147 | 157 | 176 |
| ΔE_{Host-PFCA} | 21.1 | 9.8 | 17.5 | 16.3 | 21.8 | 22.3 | 22.3 | 18.8 | 17.9 | |

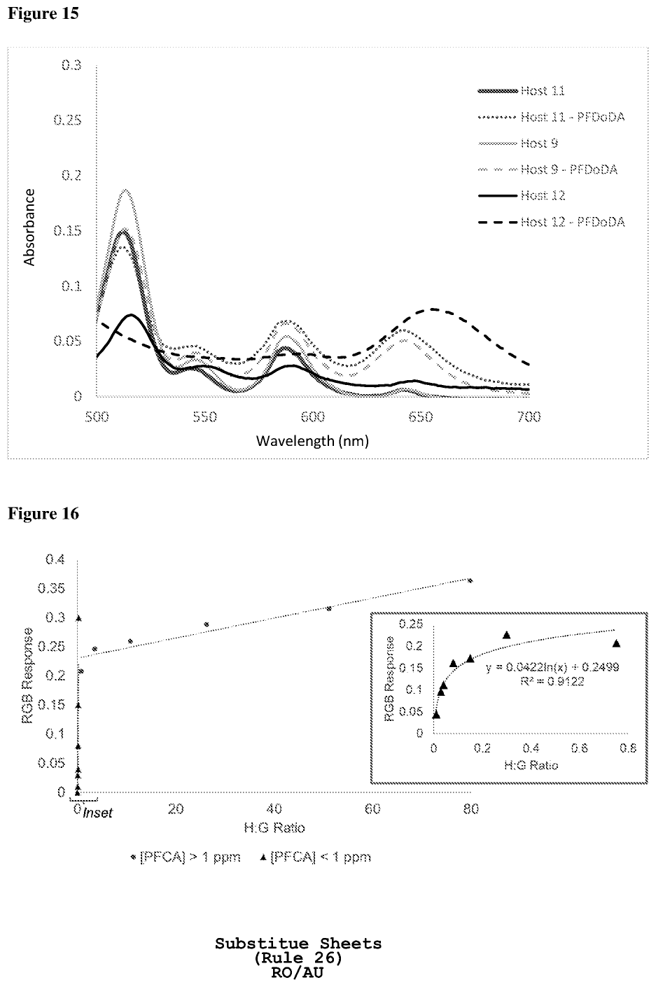

The changes in the absorption at higher wavelengths for host (12) in response to PFDoDA and long chain PFCAs are significant, suggesting there is an interaction between host (12) and long chain PFCAs. In particular, host (12) demonstrates a strong shift in the ca. 650 nm region upon binding PFDoDA while hosts (9) and (11) did not. The changes observed for hosts (9), (11), and (12) in response to PFDoDA are shown for comparison (FIG. 15).

Further spectroscopic investigations were undertaken to probe the specificities in binding from each host molecule with a single PFCA (PFOA), and each host molecule with its size matched PFCA guest (i.e., PI-BA, PFOA, PFDoDA). The association constants for PFOA for hosts (11) (logK=6.07±0.7), (9) (logK=6.25±0.8), and (12) (logK=6.22±1.3) were of similar magnitudes, indicating a comparative affinity across the hosts.

Hosts (9) and (11) demonstrated similar binding strengths with their size matched PFCAs (logK 6.12±0.9, and logK 6.25±0.9 respectively). The association constant for host (12) and PFDoDA (logK 5.68±0.5) was an order of magnitude less than that of host (9) and (11) with their equivalent size matched PFCAs, which the inventors postulate could be increased guest size adding physical hindrance. Host (12) was also modelled for secondary binding interactions in the presence of additional guest (Table 9). The secondary interactions observed for host (12) when combined with more than one molar equivalent of a PFCA guest have not been characterized, because these potential interactions were not considered to be a hindrance in the application of host (12) as a colorimetric sensor because the colorimetric response is optimal when there is more than, or approximately equal amounts of host relative to guest.

TABLE 9
Comparison of association constants for host (12) with
PFOA and PFDoDA in 1:1, 1:2 and 2:1 binding modes

| PFOA | 1 Host:1 Guest | K | K error (%) |
|---|---|---|---|
| | | 1.58 × 10⁵ | 13.4 |
| | | Log K | Log K error |
| | | 5.2 | 1.1 |

| 1 Host:2 Guest | K₁₁ | K₁₂ | K₁₁ error (%) | K₁₂ error (%) |
|---|---|---|---|---|
| | 1.49 × 10⁷ | 1.17 × 10² | 3.3 | 4.2 |
| | Log K₁₁ | Log K₁₂ | Log K₁₁ error | Log K₁₂ error |
| | 7.2 | 2.1 | 0.5 | 0.6 |

| 2 Host:1 Guest | K₁₁ | K₂₁ | K₁₁ error (%) | K₁₂ error (%) |
|---|---|---|---|---|
| | — | — | — | — |
| | Log K₁₁ | Log K₁₂ | Log K₁₁ error | Log K₁₂ error |
| | — | — | — | — |

| PFDoDA | 1 Host:1 Guest | K | K error (%) |
|---|---|---|---|
| | | 3.76 × 10⁴ | 1.9 |
| | | Log K | Log K error |
| | | 4.6 | 0.3 |

| 1 Host:2 Guest | K₁₁ | K₁₂ | K₁₁ error (% ) | K₁₂ error (%) |
|---|---|---|---|---|
| | 1.76 × 10⁶ | 7.92 × 10⁴ | 81.1 | 4.3 |
| | Log K₁₁ | Log K₁₂ | Log K₁₁ error | Log K₁₂ error |
| | 6.2 | 4.9 | 1.9 | 0.6 |
| 2 Host:1 Guest | K₁₁ | K₂₁ | K₁₁ error (%) | K₂₁ error (%) |
| | 2.37 × 10⁵ | 6.69 × 10⁴ | 5.1 | 5.9 |
| | Log K₁₁ | Log K₁₂ | Log K₁₁ error | Log K₁₂ error |
| | 5.4 | 4.8 | 0.7 | 0.8 |

### Total PFCA Detection

An image in the RGB colour space is composed of three data channels ranging from 0-255. A black object has an RGB value of (0,0,0), while a white object has an RGB value of (255,255,255). The nature of RGB colour information means that a lightening or darkening of a sample will result in a change that effects RGB values with equal direction and magnitude. That can mask changes due to colour transformations that typically impact individual colour channels independently. To better model the changes due to shifts in perceived colour, the difference between individual RGB values can be compared, or the values can be parametrized. Investigations have demonstrated that each host molecule (9), (11), and (12) can provide a colorimetric response for PFCAs of varying chain length. Although the association constants for hosts (9) and (11) suggest similar affinities for the range of tested PFCAs, the colorimetric responses show a preferential “green” change for shorter chain PFCAs.

One-way factorial ANOVA was used to examine the dependence (or independence) of two or more factors affecting the dependent variable concurrently, in the present case the change in absorption, or the change in RGB response across the tested PFCAs, or across the different hosts. This analysis was used to determine the variance in the responses of each host across the different PFCAs. The variance (σ²) in the RGB values for each sensor in response to the range of PFCAs suggests there is a difference in the colorimetric response to binding between the molecules (host (9): σ²=110, host (11): σ²=135, host (12): σ²=15). Sensor (12) had the lowest deviation and variance across the nine PFCAS; although its colour change was not the most intense, it gives a similar response across the range of PFCAs. The analysis of absorbance observed at 650 nm shows that host (12) provides a more consistent response to the range of PFCA chain lengths (σ²=9.7×10⁻⁵), which is also evidenced in the green value extracted from ImageJ analysis (σ²=14). This suggests host (12) may be a particularly practical choice for applications in determination of PFCAs in unknown samples.

### Colorimetric Calibration Charts

The host molecules were combined with known concentrations of PFOA to provide RGB colour responses, which were used to create a colorimetric calibration chart that could be trialled as an indicator for estimating the total concentration of a range of any PFCAs. Solutions of each host molecule (1.00×10⁻⁵ M) were prepared in dichloromethane. Solutions of PFOA in dichloromethane were prepared across a range of concentrations (0→16 ppm, 4.00×10⁻⁵ M). Each host (2 mL) was combined with each PFOA concentration (1 mL, final concentration 0→1.33×10⁻⁵ M) and photographed under equivalent lighting conditions. The average colour of each solution was then found using ImageJ RGB analysis, and a colour chart was produced. Plots of RGB values show that host (12) provides the most easily distinguishable colour change for any sample containing PFOA, which is supported by the larger ΔE values produced from comparison of the host and each concentration of PFOA.

At lower concentrations of PFOA, host (12) provided the largest change in RGB values, which could be observed visually, and is evidence by the comparison of ΔE values calculated between the RGB values of each host and the lowest PFOA concentration (0.7 ppm) (host (11)=4, host (9)=11, and host (12)=24). Any sample containing PFOA at concentration >0.7 ppm was distinguished from the host using RGB values but the differentiation and clustering of RGB values for samples containing PFOA demonstrate the suitability of this colorimetric response to be used as a threshold test for total PFCA contamination.

### Colorimetric Matching of a PFCA Sample—Organic

To probe the sensitivity of this technique visually, a colour chart based on the response of host (12) to known concentrations of PFOA was tested against another PFCA. The PFCA was chosen to be PFHxA, as PFOA was used for the calibration process. Samples of PFHxA (4 ppm) were prepared and combined with host (12). The average colour from triplicate experiments was compared to a calibration chart. The RGB values for host (12) were collected at six concentrations of PFOA. To make a direct comparison to the 4 ppm PFHxA sample, a colour chart was generated by fitting four colour points between the measured values for 0 and 5 ppm of PFOA. The generated RGB value for the 4 ppm PFOA was then compared to the measured RGB value for the 4 ppm PFHxA sample. When comparing the RGB values numerically using the CIE76 algorithm (Sharma et al., 2005), the ΔE values show that the 4 ppm PFOA colour would be perceived as most similar to the 4 ppm PFHxA sample (ΔE=7.3). The 3 and 5 ppm PFOA colours had ΔE values of 10.3 and 9.5 respectively, making the 4 ppm colour the most suitable match.

### Colorimetric Matching of a PFCA Sample—Biphasic

Aqueous solutions of PFOA were prepared at varying concentrations to replicate the host:guest ratio used in the dichloromethane (organic) experiment, whilst also being in range of environmentally relevant PFCA concentrations (HEPA; Mahinroosta and Senevirathna, 2020). The host (12) in dichloromethane (2.00×10⁻⁸ mol, 2 mL) was combined with aqueous solutions of PFOA (0-16 ppm, 10 mL) giving a coloured organic solution of host (12) with an aqueous phase. The colour chart was established using the RGB values collected from ImageJ analysis of the organic phase in the biphasic system. It was observed that the addition of the aqueous PFOA solution resulted in an immediate green colour change in the organic phase that rapidly returned to a colour closer to the original host solution. Research has demonstrated the aggregation behaviour of PFOA to be significant and varied across a range of concentrations (Yu et al., 2009; DeWitt, 2015; Pancras et al., 2016). It has been postulated that the observation of colour change may be due to phase partitioning between the organic host, the vial surface, and the aqueous PFCA solution (logKo/w PFOA ca. 6.44) (Rodea-Palomares et al., 2012). As such, the colour values recorded from the organic phase in the biphasic extractions differ to the entirely organic experiments, but the trends are similar.

To further probe the colorimetric response, a solution of host (12) in dichloromethane was combined with an aqueous PFHxA sample (3 ppm), shaken by hand, and allowed to settle before being photographed. The average colour was generated from ImageJ analysis. The sample was assessed visually and numerically. The ΔE values were determined for the measured PFHxA sample and each of the RGB values generated for 0-5 ppm PFOA. The ΔE values decreased with the increasing PFOA concentrations, showing that in this instance the calibration chart would overestimate the concentration of PFHxA. This could be due to the aggregation behaviours of PFCAs in aqueous solutions being influenced by the difference in ratio of organic and aqueous solutions used for the calibration process and the sample experiments. Further experiments were conducted in dichloromethane for ease and stronger colorimetric responses but could readily be adjusted to account for the expected shift in colour responses in biphasic systems.

### Parameterization of RGB Values for Determination of Any PFCA

In instances where a colour change occurs (e.g., red to green) as opposed to a change in colour intensity (e.g., red to darker red), the individual RGB channels may shift with different magnitude and direction (Firdaus et al., 2014). Thus, an increase in the average RGB value, (where

$$\overline{RGB} = \frac{( R + B + G )}{3} )$$

does not always provide adequate information for determining a colour change due to the presence of an analyte. For this reason, parametrization of the RGB values can be useful for interpreting results. The RGB response parameter can be calculated using the RGB values of the “blank” host solution (Šafranko et al., 2019) and the effective intensities of the individual RGB values of a sample (Tahir et al., 2016):

ΔR=|R_{H}−R_{S}|

ΔG=|G_{H}−G_{S}|

ΔB=|B_{H}−B_{S}|

Here, H, indicates the values for the host solution, and S, indicates the response for a sample containing a PFCA, so that ΔR, ΔG, and ΔB give the colour differences. The RGB parameter is the response due to the relative difference in the RGB intensities:

$$RGB Parameter : \frac{Δ R + Δ G + Δ B}{{R}_{H} + {G}_{H} + {B}_{H}}$$

The RGB values collected for each host with a range of known concentrations of PFOA in dichloromethane were parameterized and used to determine a relationship between concentration of analyte and RGB response values. The coefficients of determination, represented as the R² value, were higher for hosts (9) and (12) (R²=0.9598 and R²=0.8945 respectively, and host (11) R²=0.6336). The relationship between RGB response values and concentrations of PFOA could now be tested for accuracy in determining the concentration of any PFCA.

### Colorimetric Matching of Mixed PFCA Samples

The present inventors investigated the colorimetric responses for host (12) with “high” concentrations of PFCA (where [host]<[guest]), and “low” concentrations of PFCA (where [host]>[guest]). Generally, RGB response values will increase with increasing concentrations of PFCA until the host has been saturated; beyond the saturation limit, RGB response values are similar. When there was an excess of guest, the response values could only be used as a threshold indication for PFCA concentrations.

RGB response curves were established by combining solutions of host (12) (5.01×10⁻⁶ M) with solutions of PFOA (0-16 ppm, 3.86×10⁻⁵ M) in dichloromethane. Two experiments were performed; one where the host:guest ratio does not exceed 1:1 (0-40 ppb), and the other where there was an excess of guest (0-16 ppm) to mimic two potential testing situations, resulting in a logarithmic regression that levels out at a maximum determined by the quantity of host (FIG. 16). Thus, when establishing a calibration curve, the relative proportion of free host to host−PFCA complex limiting the variability of colour response must be considered for the application.

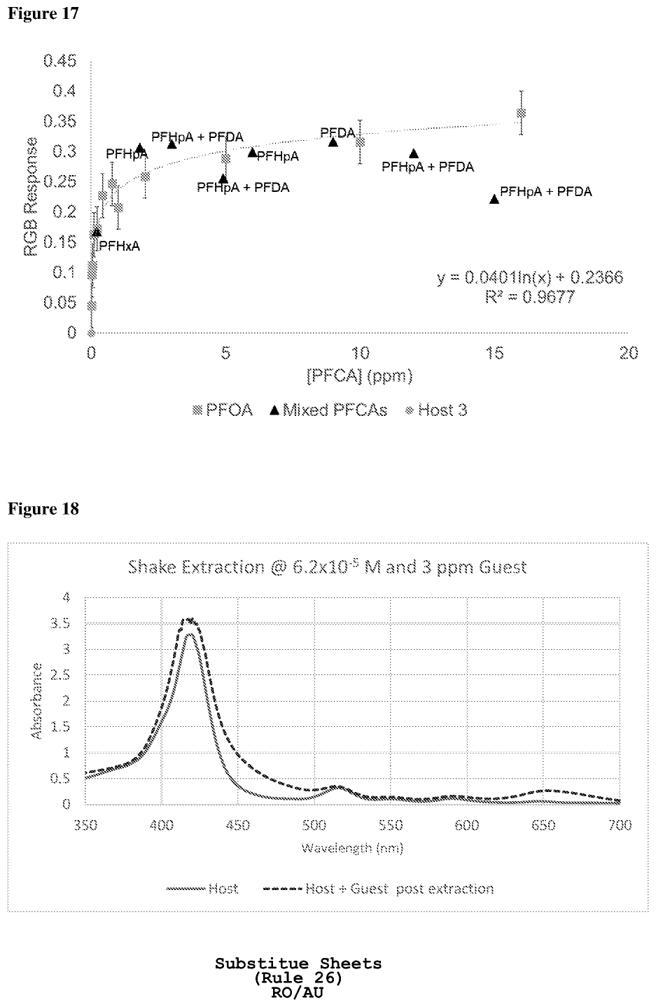

For the “high” PFCA concentration experiment, the PFOA generated calibration curve (y=0.0401 ln(x)+0.2366, R²=0.9677) (FIG. 17) begins to level out at ca. 2 ppm. Above that concentration, the RGB response values are difficult to differentiate. For example, when mixed PFCA samples of PFHxA, PFHpA, and PFDA were prepared in the 0.2-15 ppm range, the ΔE for the 15 ppm and 12 ppm PFCA samples, ΔE=2, suggests the colours would be perceived as the same (Table 10). Notably, at levels below the saturation of the host, the PFOA regression was able to predict the 0.2 ppm PFCA sample with 10% error ([PFHxA]_{measured}=0.20 ppm, [PFCA]_{predicted}=0.18 ppm). The small ΔE (0-2) for the difference between the 15 and 12 ppm samples suggests that the colours are not perceived as being different.

TABLE 10
Comparison of the ΔE values for a
15 vs 12 ppm, and 15 vs 3 ppm PFCA sample

| | R | G | B | ΔE |
|---|---|---|---|---|
| (PFHpA + PFDA) 15 ppm | 158 | 166 | 87 | |
| (PFHpA + PFDA) 12 ppm | 148 | 158 | 65 | 2 |
| (PFHpA + PFDA) 15 ppm | 158 | 166 | 87 | |
| (PFHpA + PFDA) 3 ppm | 145 | 158 | 60 | 10 |

In the “low” PFCA concentration experiment, the RGB response values had the greatest rate of change from 0-40 ppb PFOA and appeared to level out between 40-80 ppb PFOA. In that instance, the regression was fit for a host : guest ratio of 0-0.8. To probe the sensor's response to a mixture of PFCAs, samples containing combinations of PFHxA, PFHpA, and PFDA in dichloromethane (7-28 ppb) were prepared and the RGB responses were collected. The PFCA concentrations for the samples were predicted using the PFOA regression (y=0.0591 ln(x)+0.037, R²=0.9771).

The RGB response values can differentiate between 0 and 1 ppb PFOA numerically on the regression, but to determine the visual detectability, the ΔE values can be calculated. When comparing the host (12) solution with each concentration of PFOA, the smallest value of ΔE was still greater than 2, which suggests all the PFOA concentrations could technically be differentiated visually from the host (12) solution by eye. Samples from 0-5 ppb PFOA gave a colour change that could be determined as different from the starting host solution (ΔE=4-8) (Table 11). As shown in Table 11, the 1.3 ppb concentration still had a ΔE≥2, which suggests the colours can be differentiated visually. Thus, a conservative visual limit of detection of 10 ppb PFCA may be considered for an untrained observer making threshold analysis.

TABLE 11
The ΔE or “perceivable differences” in colour between
each spiked PFOA concentration and the host (12) solution

| | [PFCA] (ppb) | R | G | B | ΔE (Host − PFCA) |
|---|---|---|---|---|---|
| PFOA | 83 | 134 | 119 | 51 | 17 |
| PFOA | 41 | 130 | 112 | 50 | 15 |
| PFOA | 21 | 136 | 115 | 58 | 12 |
| PFOA | 10 | 139 | 114 | 61 | 11 |
| PFOA | 5 | 152 | 123 | 68 | 8 |
| PFOA | 3 | 153 | 125 | 75 | 6 |
| PFOA | 1 | 158 | 131 | 83 | 4 |
| Host | 0 | 166 | 134 | 91 | 0 |
| PFCAs | 7 | 144 | 107 | 70 | 11 |
| PFCAs | 28 | 140 | 102 | 62 | 12 |
| PFCAs | 14 | 142 | 106 | 64 | 11 |

The mixed PFCA samples had an average ΔE>10 when compared to the host solution and could therefore be recognized as different from the host solution visually. The concentrations estimated for the mixed PFCA samples using the PFOA regression also suggested the limit of detection was above 10 ppb. The lowest concentration PFCA sample (7 ppb) was predicted to be ca. 11 ppb (58% error) from the PFOA regression, whilst the higher concentration mixed PFCA samples (14.1 and 28.8 ppb) were predicted to be ca. 16 and 23 ppb (17 and 18% error respectively). Thus, host (12) may be coupled with a phone camera to estimate mixed PFCA concentrations from RGB data with <20% error, or used visually for threshold detection of total PFCA concentrations above 10 ppb.

### Example 4. Extraction Tests

### Filter Paper Tests

Sensor molecule (9) was dissolved in dichloromethane and filter papers were soaked in the solution. The papers were removed and dried by evaporation. The paper was then dipped into aqueous solutions of PFOA or aqueous solutions were dropped onto the paper. A colour change was discernible. The aqueous solutions of PFOA tested were in the <5 ppm range.

### Aqueous Extraction Tests

Aqueous solutions of PFOA were prepared and combined with an organic phase of sensor molecule (9) in dichloromethane. Colour change observations at various sensor concentrations are described in Table 12.

TABLE 12
Colour change observations at various sensor concentrations

| | | | Mole | | |
|---|---|---|---|---|---|
| [Sensor (10)] | [PFOA] in | Volume | equivalents | | PFOA |
| in DCM (M) | water (M) | equivalents | (H:G) | Observations | (~ppm) |
| 2.2 × 10⁻³ | 5.5 × 10⁻⁴ | 1:1 | 4:1 | Strong colour change | 228 |
| 2.2 × 10⁻⁴ | 5.5 × 10⁻⁴ | 1:1 | 2:5 | Strong colour change | 228 |
| 2.2 × 10⁻⁵ | 5.5 × 10⁻⁴ | 1:1 | 1:25 | Strong colour change | 228 |
| 1.1 × 10⁻⁶ | 5.5 × 10⁻⁴ | 1:1 | 1:500 | Some colour change, | 228 |
| | | | | difficult to detect | |
| | | | | without comparison | |

### “Shake” Extraction Vial Trials

A solution of sensor (9) was dissolved in dichloromethane (6.2×10⁻⁵ M) and combined with aqueous solutions of PFOA (3 ppm). The vials are capped and agitated. The organic phase was collected for UV-Vis analysis as shown in FIG. 18.

### Soil Tests

Soil was taken front the top 15 cm of the garden bed outside the Chemistry Building at the University of Tasmania and dried in the oven at 40° C. for approximately 60 hours. This soil was then spiked with 10 ppm PFOA: Two 50 g dried soil samples were transferred into two separate 1 L polypropylene containers. One sample was combined with a dichloromethane-PFOA solution (200 mL of 6×10⁻⁶ M PFOA in DCM) and the other was combined with only dichloromethane (200 mL). The solutions were left to evaporate. 5 g of the resulting samples was transferred to high density polyethylene tubes.

Combinations of sensor molecule (9) in different organic solvents were added to spiked and control samples, and the results were compared. Water was also added to see if a biphasic system was beneficial for extraction from soil.
A=20 mL dichloromethane+medium chain sensor (9)
B=10 mL of water-->20 mL dichloromethane+medium chain sensor (9)
C=20 mL ethyl acetate+medium chain sensor (9)
D=20 mL toluene+medium chain sensor (9)

This experiment allowed for the efficiency of extraction using different solvents to be probed alongside their applicability as a colorimetric indicator for visual assessment of PFAS contamination. The results of these soil extractions showed that the dichloromethane provided the most obvious colour change in the presence of the soil matrices. If the soil was composed of both flocculant and sediment, in some cases the biphasic water layer aided in making the organic phase clearer by separating the solids from the host sensor.

Dried soil samples (5 g) were also combined with 20 mL of host (9) in dichloromethane and shaken to show a colour change observable in the solvent. Even with no pre-treatment or pre-concentration, a colour change in host molecule (9) was discernible by the naked eye.

### Example 5. GenX™ Binding Studies

The ability of porphyrin derivatives of the present invention to bind GenX™ (the ammonium salt of hexafluoropropylene oxide dimer acid (HFPO-DA) fluoride) was studied using UV-Vis spectroscopy.

### Medium Chain Host (9)+GenX or PFOA

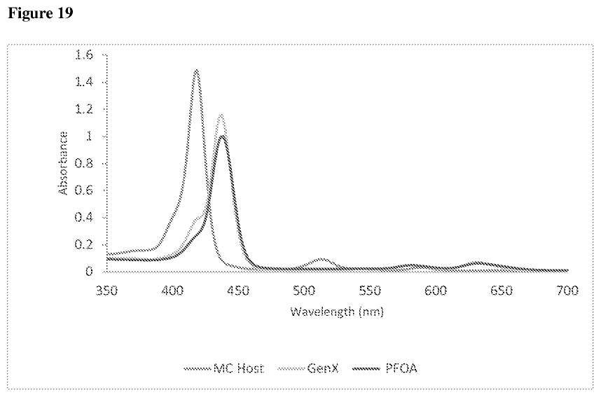

A solution of medium chain host (9) (4.46×10⁻⁶ M) in dichloromethane was prepared as the working host solution. A solution of PFOA (4.43×10⁻⁴ M) was prepared in the host solution. A solution of GenX™ (4.43×10⁻⁴ M) was prepared in the host solution. The host solution (2 mL) was added into a glass cuvette (1 cm path length) and a UV-visible spectrum was collected. An aliquot of the PFOA guest solution (100 μL) was added and the UV-visible spectrum was collected again. This procedure was repeated for the GenX™ guest solution (FIG. 19).

### Medium Chain Amide Host (9)+GenX Water Vapour (Preliminary Experiment)

A solution of medium chain host (9) (2.20×10⁻⁵ M) in dichloromethane was prepared. Whatman 4 filter papers were soaked/dipped in the host solution and left to air dry on a watch glass. An aqueous solution of GenX™ (1 ppm, 3.03×10⁻⁶ M) was prepared in a glass volumetric flask (1 L). The volumetric flask stopper was removed, and the dried host-doped paper was laid on top the opening. A colour change developed immediately.

### Medium Chain Amide Host+GenX Vapour (Preliminary Experiments)

An aqueous solution of GenX™ (1 ppm, 3.03×10⁻⁶ M) was prepared in a glass volumetric flask (1 L). A solution of medium chain host (2.20×10⁻⁵ M) in dichloromethane was prepared. Whatman 4 filter papers were soaked/dipped in the host solution and left to air dry on a watch glass. The filter papers were cut into strips and adhered to the lids of HDPP tubes. Aqueous samples of GenX™ were diluted to 1 ppm, 1 ppb and 1 ppt and poured into the HDPP tubes. The caps were immediately replaced, and the tubes were left for an hour. A green colour was visible for papers in the 1 ppm and 1 ppb samples.

### Short Chain Amide Sensor+GenX CDCl₃ Preliminary Experiment

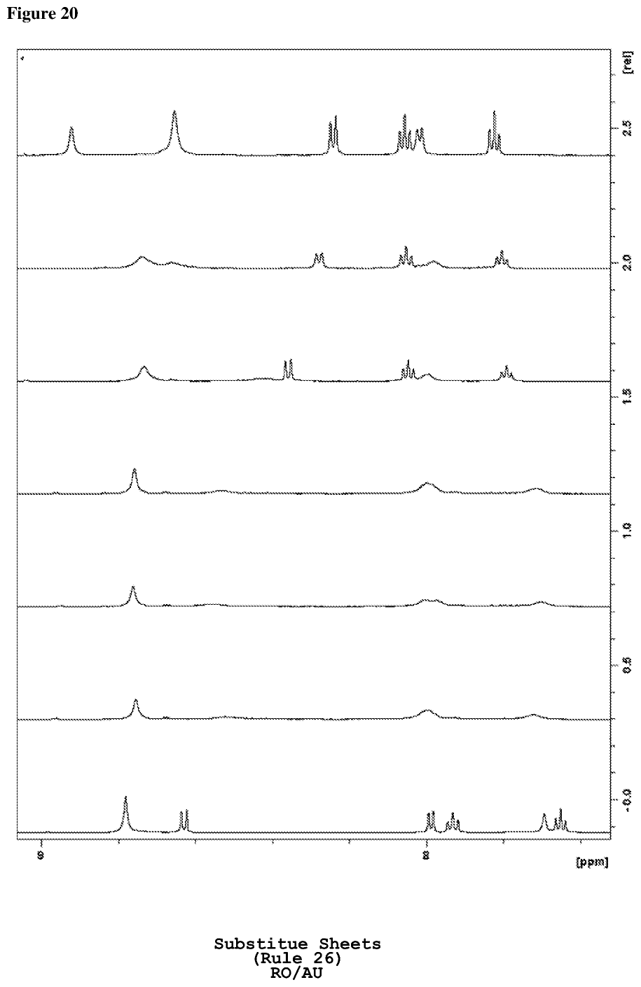

A solution of short chain amide sensor (0.002 g, 1.37×10⁻⁶ mol) was prepared in deuterated chloroform (2 mL, 6.85×10⁻⁴ M). A solution of GenX (0.002 g, 6.06×10⁻⁶ mol) was prepared in the host solution (1.5 mL, 4.04×10⁻³ M). An aliquot (0.5 mL) of pure host solution was transferred to an NMR tube. A ¹H NMR proton spectrum was collected, and aliquots of guest solution (20 μL) were added and sequentially analysed. ¹H NMR spectra showed shifts in amide porphyrin derivative in response to addition of GenX™ (FIG. 20).

### GenX, Water and HDPP Tube Experiment

A solution of medium chain amide host (2.04×10⁻⁵ M) in dichloromethane was added to an aqueous solution of GenX™ (5 ppm, 1.51×10⁻⁵ M 50 mL) in a volumetric flask. The host solution immediately became a vibrant green colour. Both phases were then transferred via pouring to a HDPP tube. The green colour persisted initially but began to reduce with time. After 24 hours there was a significant reduction in the intensity of the green colour.

### REFERENCES

Alexander, B. H.; Olsen, G. W., 2007. Bladder cancer in perfluorooctanesulfonyl fluoride manufacturing workers. Annals of epidemiology, 17 (6), 471-8.
Bales, B. L.; Zana, R., 2004. Cloud Point of Aqueous Solutions of Tetrabutylammonium Dodecyl Sulfate Is a Function of the Concentration of Counterions in the Aqueous Phase. Langmuir, 20 (5), 1579-1581.
Castillo, P. M., Fernández-Acejo, F. J., Carnerero, J. M., Prado-Gotor, R., Jimenez-Ruiz, A., 2021. Colorimetric, Naked-Eye Detection of Lysozyme in Human Urine with Gold Nanoparticles. Nanomaterials 11, 612.
Emmett, E. A.; Shofer, F. S.; Zhang, H.; Freeman, D.; Desai, C.; Shaw, L. M., 2006. Community exposure to perfluorooctanoate: relationships between serum concentrations and exposure sources. J Occup Environ Med, 48 (8), 759-70.
Eriksen, K. T.; Sørensen, M.; McLaughlin, J. K.; Lipworth, L.; Tjønneland, A.; Overvad, K.; Raaschou-Nielsen, O., 2009. Perfluorooctanoate and Perfluorooctanesulfonate Plasma Levels and Risk of Cancer in the General Danish Population. JNCI: Journal of the National Cancer Institute, 101 (8), 605-609.
Firdaus, M. L., Alwi, W., Trinoveldi, F., Rahayu, I., Rahmidar, L., Warsito, K., 2014. Determination of Chromium and Iron Using Digital Image-based Colourimetry. Procedia Environmental Sciences 20, 298-304.
Gallagher, S.R., 2014a. Digital Image Processing and Analysis with ImageJ. Current Protocols Essential Laboratory Techniques 9, A.3C.1-A.3C.29.
Gallagher, S.R., 2014b. Digital Image Processing and Analysis with ImageJ. Current Protocols Essential Laboratory Techniques 9.
Göckener, B., Weber, T., Rüdel, H., Bücking, M., Kolossa-Gehring, M., 2020, Human biomonitoring of per- and polyfluoroalkyl substances in German blood plasma samples from 1982 to 2019. Environ. Int., 145, 106123.
‘PFAS National Environmental Management Plan Version 2.0’, Heads of EPA Australia and New Zealand 2020’
Hibbert, D. B., Thordarson, P., 2016. The death of the Job plot, transparency, open science and online tools, uncertainty estimation methods and other developments in supramolecular chemistry data analysis. Chem. Commun. 52, 12792-12805.
Huset, C. A.; M. Barry, K., 2018. Quantitative determination of perfluoroalkyl substances (PFAS) in soil, water, and home garden produce. MethodsX, 5, 697-704.
Inoue, K.; Okada, F.; Ito, R.; Kato, S.; Sasaki, S.; Nakajima, S.; Uno, A.; Saijo, Y.; Sata, F.; Yoshimura, Y.; Kishi, R.; Nakazawa, H., 2004. Perfluorooctane sulfonate (PFOS) and related perfluorinated compounds in human maternal and cord blood samples: assessment of PFOS exposure in a susceptible population during pregnancy. Environmental health perspectives, 112 (11), 1204-1207.
Jones, P. D.; Hu, W.; De Coen, W.; Newsted, J. L.; Giesy, J. P., 2003. Binding of perfluorinated fatty acids to serum proteins. Environmental toxicology and chemistry, 22 (11), 2639-49.
Kölmel, D. K.; Rudat, B.; Braun, D. M.; Bednarek, C.; Schepers, U.; Bräse, S., 2013. Rhodamine F: a novel class of fluorous ponytailed dyes for bioconjugation. Organic & Biomolecular Chemistry, 11 (24), 3954.
Lee, C.-H.; Lee, S.; Yoon, H.; Jang, W.-D., 2011. Strong Binding Affinity of a Zinc-Porphyrin-Based Receptor for Halides through the Cooperative Effects of Quadruple C—H Hydrogen Bonds and Axial Ligation. Chemistry—A European Journal, 17 (49), 13898-13903.
Lindsey, J., 2002. Increased yield of a desired isomer by equilibriums displacement on binding to silica gel, applied to meso-tetrakis(o-aminophenyl)porphyrin. Journal of Organic Chemistry, 45, 5215.
Mahinroosta, R., Senevirathna, L., 2020. A review of the emerging treatment technologies for PFAS contaminated soils. J Environ Manage 255, 109896.
Menesatti, P., Angelini, C., Pallottino, F., Antonucci, F., Aguzzi, J., Costa, C., 2012. RGB Colour Calibration for Quantitative Image Analysis: The “3D Thin-Plate Spline” Warping Approach. Sensors (Basel, Switzerland), 12, 7063-7079.
Pancras, T., Schrauwen, G., Held, T., Baker, K., Ross, I., Slenders, H., Smith, J. W. N., Beuthe, B., Dunk, M., Demeure, S., Carmona, J. M. M., Medve, A., Spence, M. J., 2016. Environmental fate and effects of polyand perfluoroalkyl substances (PFAS) in: (STF/33), C.S.a.G.T. (Ed.). Concawe, pp. 1-121.
Phuangsaijai, N., Jakmunee, J., Kittiwachana, S., 2021. Investigation into the predictive performance of colorimetric sensor strips using RGB, CMYK, HSV, and CIELAB coupled with various data preprocessing methods: a case study on an analysis of water quality parameters. Journal of Analytical Science and Technology 12.
Rodea-Palomares, I., Leganes, F., Rosal, R., Fernandez-Pinas, F., 2012. Toxicological interactions of perfluorooctane sulfonic acid (PFOS) and perfluorooctanoic acid (PFOA) with selected pollutants. J. Hazard. Mater. 201-202, 209-218.
Šafranko, S., Živković, P., Stanković, A., Medvidović-Kosanović, M., Széchenyi, A., Jokić, S., 2019. Designing ColourX, Image Processing Software for Colorimetric Determination of Concentration, To Facilitate Students' Investigation of Analytical Chemistry Concepts Using Digital Imaging Technology. J. Chem. Educ., 96, 1928-1937.
Sharma, G., Wu, W., Dalal, E. N., 2005. The CIEDE2000 colour-difference formula: Implementation notes, supplementary test data, and mathematical observations. Colour Research & Application, 30, 21-30.
Sorrell, T. N.; Bump, C. M.; Jordan, J., 2007. (Dioxygen)(N-Methylimidazole)[All-Cis)-5 10,15,20-Tetrakis[2-(2,2-Dimethylpropionamido)-Phenyl]Porphyrinato(2-)]Iron(II). In Inorganic Syntheses; pp 161-169.
Tahir, H.E., Xiaobo, Z., Xiaowei, H., Jiyong, S., Mariod, A. A., 2016. Discrimination of honeys using colorimetric sensor arrays, sensory analysis and gas chromatography techniques. Food Chem., 206, 37-43.
Thordarson, P., 2011. Determining association constants from titration experiments in supramolecular chemistry. Chemical Society Reviews, 40 (3), 1305-1323.
Usachova, N.; Leitis, G.; Jirgensons, A.; Kalvinsh, I., 2010. Synthesis of Hydroxamic Acids by Activation of Carboxylic Acids with N,N′-Carbonyldiimidazole: Exploring the Efficiency of the Method. Synthetic Communications, 40 (6), 927-935.
Xu, H. J.; Zhao, Y. Q.; Zhou, X. F., 2011. Palladium-catalyzed Heck reaction of aryl chlorides under mild conditions promoted by organic ionic bases. J Org Chem, 76 (19), 8036-41.
Yu, Q., Zhang, R., Deng, S., Huang, J., Yu, G., 2009. Sorption of perfluorooctane sulfonate and perfluorooctanoate on activated carbons and resin: Kinetic and isotherm study. Water Res. 43, 1150-1158.

1. A porphyrin derivative of Formula (I) or (II) 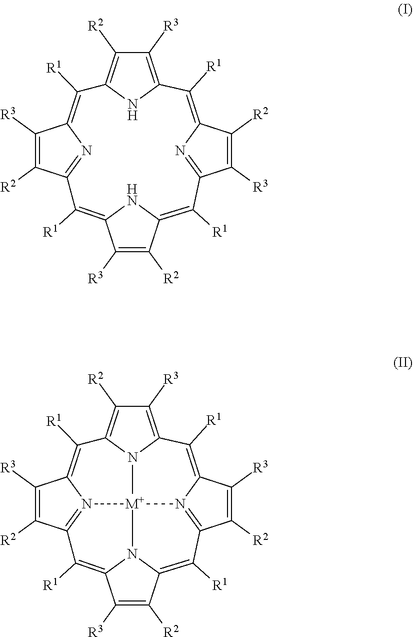
or a conformational isomer, stereoisomer and/or salt thereof, wherein
each R¹ is C₆₋₁₀ aryl substituted with one or two —A—R^{X} groups or C₂₋₁₂ heteroaryl substituted with one or two —A—R^{X} groups, wherein
A is an anion binding group selected from the group consisting of an amide, NR′ (wherein R′ is selected from H and C₁₋₄alkyl), urea, thiourea and triazole;
R^{X} is a straight chain, branched chain or cyclic C₃₋₁₅ poly- or perfluoroalkyl group or C₃₋₁₅ poly- or perfluoroalkylether;
each R² and R³ is independently selected from the group consisting of H, halo, CF₃, NR″R″′ (wherein R″ and R″′ are independently selected from H and C₁₋₄ alkyl), NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₆ alkoxy, C(O)C₁₋₆ alkyl, C(O)OC₁₋₆ alkyl, C₆₋₁₀ aryl optionally substituted with one or more R^{Y} groups, C₂₋₁₂ heteroaryl optionally substituted with one or more R^{Y} groups and tri(C₆₋₁₀ aryl)borane optionally substituted with one or more R^{Y} groups or wherein R² and R³ together with the carbon atoms to which they are attached form a phenyl ring optionally substituted with one or more R^{Y} groups,
wherein each R^{Y} is independently selected from the group consisting of halo, CF₃, NR″R″′ (wherein R″ and R″′ are independently selected from H and C₁₋₄ alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl; and
M⁺ is a metal ion,
with the proviso that the porphyrin derivative of Formula (I) or Formula (II) is not:
α,α,α,α-5,10,15,20-tetrakis(heptafluorobutyrami dophenyl)porphyrin;
α,α,α,α-5,10,15,20-tetrakis(2-(pentadecafluorooctanoylamino)phenyl)porphyrin;
α,α,α,β-5,10,15,20-tetrakis(2-(pentadecafluorooctanoylamino)phenyl)porphyrin;
α,α,β,β-5, 10,15,20-tetrakis(2-(pentadecafluorooctanoylamino)phenyl)porphyrin;
6,20-Bis[4-(tridecafluoroheptanoylamino)phenyl]tetrabenzoporphyrin;
6,13,20,27-tetrakis[4-(tridecafluoroheptanoylamino)-phenyl]tetrabenzoporphyrin;
5,10,15,20-tetrakis(2-(2,3,3,3-tetrafluoro-2-(heptafluoropropoxy)propanoylamino)phenyl)porphyrin;
α,α,α,α-5,10,15,20-tetrakis(heptafluorobutyrami dophenyl)porphyrin;
6,20-Bis[4-(tridecafluoroheptanoylamino)phenyl]tetrabenzoporphyrin platinum;
6,13,20,27-tetrakis[4-(tridecafluoroheptanoylamino)-phenyl]tetrabenzoporphyrin platinum;
5,10,15,20-tetrakis(2-(2,3,3,3-tetrafluoro-2-(heptafluoropropoxy)propanoylamino)phenyl)porphyrin cobalt; or
α,α,α,α-5,10,15,20-tetrakis(heptafluorobutyramidophenyl)porphyrin cobalt.

2. The porphyrin derivative according to claim 1, which has a picket fence conformation.

3. The porphyrin derivative according to claim 1, wherein R¹ is a phenyl group substituted with one —A—R^{X} group.

4. The porphyrin derivative according to claim 1, wherein A is selected from the group consisting of an amide, urea and triazole.

5. The porphyrin derivative according to claim 1, wherein R^{X} is a straight chain C₃₋₁₅ poly- or perfluoroalkyl group or C₃₋₁₅ poly- or perfluoroalkylether group.

6. The porphyrin derivative according to claim 1, wherein R^{X} is a straight chain C₇₋₈ poly- or perfluoroalkyl group or C₇₋₈ poly- or perfluoroalkylether group.

7. The porphyrin derivative according to claim 1, wherein R^{X} is a straight chain C₉₋₁₂ poly- or perfluoroalkyl group or C₉₋₁₂ poly- or perfluoroalkylether group.

8. The porphyrin derivative according to claim 1, wherein R^{X} is a perfluoroalkyl group.

9. The porphyrin derivative according to claim 1, wherein each R² and R³ is H.

10. The porphyrin derivative according to claim 1, wherein each R¹ is phenyl substituted with one —A—R^{X} group, A is an amide, urea or triazole, R¹ is a straight chain C₃₋₁₅ perfluoroalkyl group, each R² and R³ is H, and wherein the porphyrin derivative has a picket fence conformation.

11. A porphyrin derivative according to claim 1, having structure of Formula (IA) or (IIA) 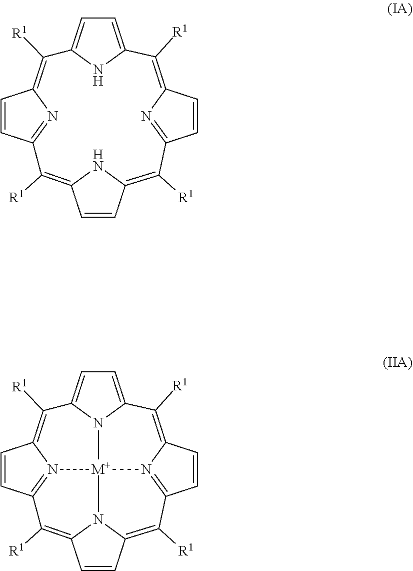
or a conformational isomer, stereoisomer and/or salt thereof, wherein
each R¹ is a phenyl group substituted with one —A—R^{X} group, wherein
A is an anion binding group selected from the group consisting of an amide, NR′ (wherein R′ is selected from H and C₁₋₄ alkyl), urea, thiourea and triazole;
R^{X} is a straight chain, branched chain or cyclic C₃₋₁₅ poly- or perfluoroalkyl group; and
M⁺ is a metal ion,
with the proviso that the porphyrin derivative of Formula (IA) or Formula (IIA) is not:
α,α,α,α-5,10,15,20-tetrakis(heptafluorobutyramidophenyl)porphyrin;
α,α,α,α-5,10,15,20-tetrakis(2-(pentadecafluorooctanoylamino)phenyl)porphyrin;
α,α,α,β-5,10,15,20-tetrakis(2-(pentadecafluorooctanoylamino)phenyl)porphyrin;
α,α,β,β-5, 10,15,20-tetrakis(2-(pentadecafluorooctanoylamino)phenyl)porphyrin;
5,10,15,20-tetrakis(2-(2,3,3,3-tetrafluoro-2-(heptafluoropropoxy)propanoylamino)phenyl)porphyrin;
α,α,α,α-5,10,15,20-tetrakis(heptafluorobutyramidophenyl)porphyrin;
5,10,15,20-tetrakis(2-(2,3,3,3-tetrafluoro-2-(heptafluoropropoxy)propanoylamino)phenyl)porphyrin cobalt; or
α,α,α,α-5,10,15,20-tetrakis(heptafluorobutyramidophenyl)porphyrin cobalt.

12. The porphyrin derivative according to claim 1, selected from the group consisting of:

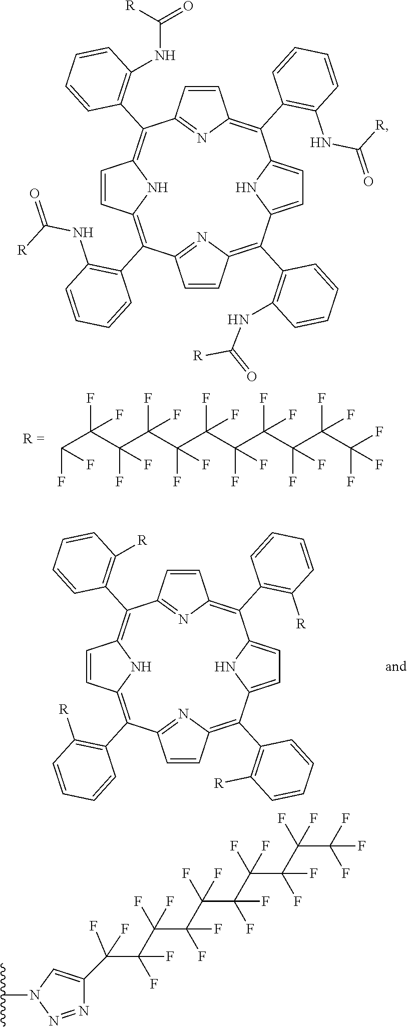

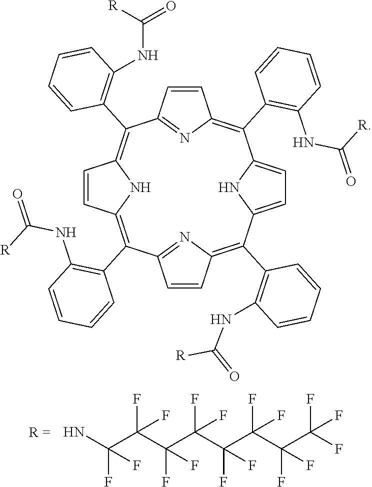

13. A method of detecting a poly- or perfluoroalkyl substance (PFAS) in a sample comprising the steps of:
(i) contacting a sample suspected of containing a PFAS with a porphyrin derivative of Formula (I) or Formula (II) 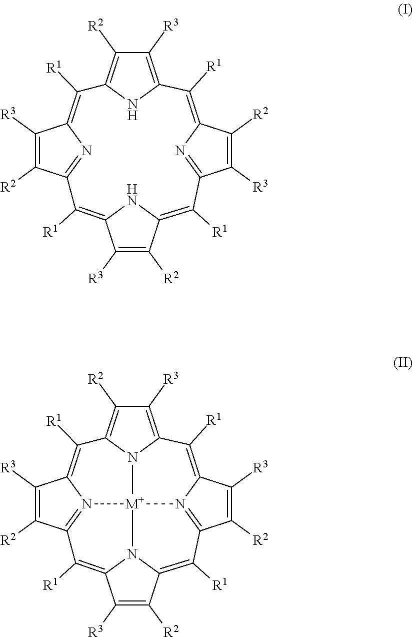
or a conformational isomer, stereoisomer and/or salt thereof, wherein
at least one R¹ is —A—R^{X}, C₆₋₁₀ aryl substituted with one or two —A—R^{X} groups or C₂₋₁₂ heteroaryl substituted with one or two A—R^{X} groups, wherein
each A is independently an anion binding group selected from the group consisting of a carbamate, toluene sulfonamide, amidourea, amide, NR′ (wherein R′ is selected from H and C₁₋₄ alkyl), ammonium, urea, thiourea, amido thiourea, guanidinium, squaramide and C₂₋₁₂ N-heteroaryl or a salt thereof optionally substituted with one or more R^{Y} groups;
each R^{X} is independently a straight chain, branched chain or cyclic C₁₋₂₀ poly- or perfluoroalkyl group, C₂₋₂₀ poly- or perfluoroalkenyl group, C₂₋₂₀ poly- or perfluoroalkynyl group or C₂₋₂₀ poly- or perfluoroalkylether group; and
the remaining R¹ groups are independently selected from the group consisting of H, halo, CF₃, NH₂, NO₂, CN, C₁₋₆ alkyl, C₁₋₆ alkenyl, C₁₋₆ alkynyl, C₃₋₁₀ cycloalkyl, C₅₋₁₀ cycloalkenyl, C₂₋₅ heterocycloalkyl, haloC₁₋₄alkyl, C₁₋₄ alkoxy, C(O)C₁₋₄ alkyl, C(O)OC₁₋₄ alkyl, C₆₋₁₀ aryl optionally substituted with one or more R^{Y} groups and C₂₋₁₂ heteroaryl optionally substituted with one or more R^{Y} groups,
each R² or R³ is independently selected from the group consisting of H, halo, CF₃, NR″R″′ (wherein R″ and R″′ are independently selected from H and C₁₋₄ alkyl), NO₂, CN, C₁₋₆ alkyl, C₂₋₆ alkenyl, C₂₋₆ alkynyl, C₃₋₁₁) cycloalkyl, C₅₋₁₀ cycloalkenyl, C₂₋₅ heterocycloalkyl, haloC₁₋₄ alkyl, C₁₋₆ alkoxy, C(O)C₁₋₆ alkyl, C(O)OC₁₋₆ alkyl, C₆₋₁₀ aryl optionally substituted with one or more R^{Y} groups, C₂₋₁₂ heteroaryl optionally substituted with one or more R^{Y} groups, and tri(C₆₋₁₀aryl)borane optionally substituted with one or more R^{Y} groups or wherein R² and R³ together with the carbon atoms to which they are attached form a phenyl ring optionally substituted with one or more R^{Y} groups,
wherein each R^{Y} is independently selected from the group consisting of halo, CF₃, NR″R″′ (wherein R″ and R″′ are independently selected from H and C₁₋₄ alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl; and
M⁺ is a metal ion,
(ii) detecting a photophysical change of the porphyrin derivative,
wherein a photophysical change of the porphyrin derivative indicates that the sample contains a PFAS.

14. The method according to claim 13, wherein the photophysical change is a visual colour change.

15. The method according to claim 13, wherein the sample is an environmental sample or a biological sample.

16. The method according to claim 15, wherein the environmental sample is a water sample or a soil sample.

17. The method according to claim 15, wherein the biological sample is blood serum sample.

18. The method according to claim 13, wherein R^{X} is complementary in carbon chain size to the PFAS suspected of being present in the sample.

19. The method according to claim 13, wherein R^{X} is a straight chain C₇₋₈ alkyl perfluoroalkyl group.

20. The method according to claim 13, wherein step (ii) comprises detecting a photophysical change visually, by UV-Vis spectroscopy, fluorescence spectroscopy or colour space analysis.

21. The method according to claim 13, wherein step (i) comprises an aqueous extraction, dipstick test or soil extraction test.

22. (canceled)

23. A host−guest complex comprising a porphyrin derivative of Formula (I) or Formula (II) and a poly- or perfluoroalkyl substance (PFAS) 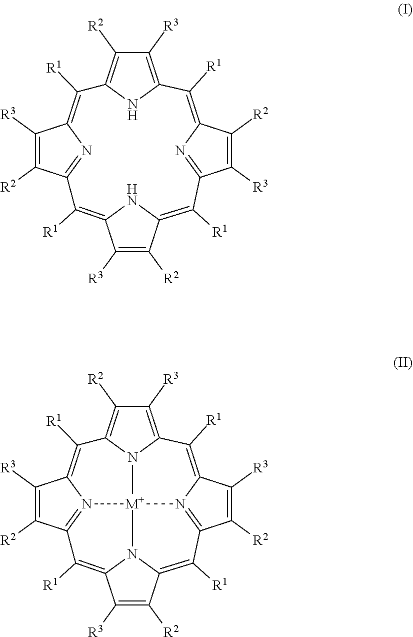
or a conformational isomer, stereoisomer and/or salt thereof, wherein
at least one R¹ is —A—R^{X}, C₆₋₁₀ aryl substituted with one or two —A—R^{X} groups or C₂₋₁₂ heteroaryl substituted with one or two A—R^{X} groups, wherein
each A is an anion binding group independently selected from the group consisting of a carbamate, toluene sulfonamide, amidourea, amide, NR′ (wherein R′ is selected from H and C₁₋₄ alkyl), ammonium, urea, thiourea, amido thiourea, guanidinium, squaramide and C₂₋₁₂ N-heteroaryl or a salt thereof optionally substituted with one or more R^{Y} groups;
each R^{X} is a straight chain, branched chain or cyclic C₁₋₂₀ poly- or perfluoroalkyl group, C₂₋₂₀ poly- or perfluoroalkenyl group, C₂₋₂₀ poly- or perfluoroalkynyl group or C₂₋₂₀ poly- or perfluoroalkylether group; and
the remaining R¹ groups are independently selected from the group consisting of H, halo, CF₃, NH₂, NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₄ alkoxy, C(O)C₁₋₄ alkyl, C(O)OC₁₋₄ alkyl, C₆₋₁₀ aryl optionally substituted with one or more R^{Y} groups and C₂₋₁₂ heteroaryl optionally substituted with one or more R^{Y} groups,
each R² or R³ is independently selected from the group consisting of H, halo, CF₃, NR″R″′ (wherein R″ and R″′ are independently selected from H and C₁₋₄ alkyl), NO₂, CN, C₁₋₆ alkyl, C₂₋₆ alkenyl, C₂₋₆ alkynyl, C₃₋₁₀ cycloalkyl, C₅₋₁₀ cycloalkenyl, C₂₋₅ heterocycloalkyl, haloC₁₋₄ alkyl, C₁₋₆ alkoxy, C(O)C₁₋₆ alkyl, C(O)OC₁₋₆ alkyl, C₆₋₁₀ aryl optionally substituted with one or more R^{Y} groups, C₂₋₁₂ heteroaryl optionally substituted with one or more R^{Y} groups, and tri(C₆₋₁₀ aryl)borane optionally substituted with one or more R^{Y} groups or wherein R² and R³ together with the carbon atoms to which they are attached form a phenyl ring optionally substituted with one or more R^{Y} groups,
wherein each R^{Y} is independently selected from the group consisting of halo, CF₃, NR″R″′ (wherein R″ and R″′ are independently selected from H and C₁₋₄ alkyl), NO₂, CN, C₁₋₄ alkyl, C₁₋₄ alkenyl, C₁₋₄ alkynyl, haloC₁₋₄ alkyl, C₁₋₄ alkoxy, C(O)C₁₋₄ alkyl and C(O)OC₁₋₄ alkyl; and
M⁺ is a metal ion.
